(19) United States
(12) Patent Application Publication
(10) Pub. No.: US 2009/0235370 A1
(43) Pub. Date: Sep. 17, 2009

(54) SECRETED LUCIFERASE FOR EX VIVO MONITORING OF IN VIVO PROCESSES

(75) Inventor: Bakhos A. Tannous, Malden, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, BOSTON, MA (US)

(21) Appl. No.: 12/355,055

(22) Filed: Jan. 16, 2009

Related U.S. Application Data
(60) Provisional application No. 61/021,402, filed on Jan. 16, 2008.

(51) Int. Cl. A01K 67/027; C12Q 1/66; C12N 5/06

(52) U.S. Cl. 800/3; 435/8; 435/325; 800/13; 800/14

(57) ABSTRACT

The present invention generally relates to a methods, compositions and assays for real-time monitoring of the progression of a disease, such as a cancer in a subject, by measuring the level of bioluminescence signal in a biological sample obtained from a subject, where the bioluminescence signal is from a secreted luciferase protein expressed by a cell or tissue in the subject. One aspect of the present invention relates to administering to a subject a nucleic acid encoding a secreted luciferase, and in some embodiments, a disease or a diseased tissue such as tumor cells expresses the secreted luciferase protein, which is monitored in a biological sample, such as blood or urine obtained from a subject. One aspect of the invention relates to analysis of a secreted luciferase protein by measuring the level in a biological sample obtained from the subject without the need for invasive monitoring procedures.

### CROSS REFERENCE TO RELATED APPLICATIONS

This application claims priority under 35 U.S.C. 119(e) of U.S. Provisional Patent Application Ser. No. 61/021,402 filed 16 Jan. 2008, the contents of which are incorporated herein by reference in their entirety.

### GOVERNMENT SUPPORT

This invention was made with Government support under P50 CA86355-04 and IK99CA126839-01 awarded by the National Cancer Institute. The Government may have certain rights in the invention.

### FIELD OF THE INVENTION

The present invention relates generally to materials and methods for a non-invasive real-time method for monitoring the presence of a disease in a subject, and more particularly to a method to monitor the presence and progression of a cancer in a subject by using a secreted luciferase protein which functions as a reporter for the presence of cancer cells, and where the secreted luciferase protein is measured in a biological sample.

### BACKGROUND

Luciferases are widely used to monitor various biological processes. Luciferase-mediated bioluminescence imaging has served as a reporting tool for monitoring various biological processes in vitro and in vivo in different fields³, including immunology⁴ oncology⁵, virology⁶, and neuroscience⁷. After systemic substrate injection, a charge coupled device (CCD) camera can be used to localize the luciferase photon signals in vivo. Recently, a naturally secreted luciferase from the marine copepod Gaussia princeps (Gaussia luciferase, Gluc) has been characterized and found it to be over 2000-fold more sensitive than firefly and Renilla reniformis luciferases and 20,000-fold more sensitive than the secreted alkaline phosphatase (SEAP) when expressed in mammalian cells^{8,9}. Gluc expression levels can be easily quantified in cell-free, conditioned medium by adding its substrate coelenterazine and measuring emitted photons using a luminometer. After systemic substrate injection, a cooled charge-coupled device (CCD) camera can be used for the localization of the luciferase photon signals in vivo. In addition, the naturally secreted and non-ATP dependent Gaussia luciferase (Gluc) expression levels can be easily quantified in cell-free conditioned medium by adding its substrate coelenterazine and measuring emitted photons using a luminometer.

### SUMMARY

The present invention relates to methods and compositions for monitoring in vivo biological processes by measuring the level of a secreted luciferase in ex vivo samples. As disclosed herein, the inventors have discovered that a naturally secreted Gaussia luciferase (herein referred to as “Gluc”) as a highly sensitive reporter for quantitative assessment of cells in vivo by measuring its levels in blood. Using this secreted Gaussia luciferase, the inventors have discovered a method to measure an in vivo biological process by measuring secreted luciferase in a biological sample, for example a blood sample.

As used herein, the term “Gluc blood assay” refers to measuring a secreted luciferase in a blood sample. Accordingly, the inventors have discovered a method to measure a secreted luciferase in a biological sample which is useful to complement in vivo bioluminescence imaging which typically provides information such as location of the signal in the body and provides a multifaceted assessment of cell viability, proliferation and location in experimental disease and therapy models. Since Gluc is secreted from mammalian cells in culture⁹, the inventors demonstrated Gluc can be secreted into the blood of animals harboring cells expressing this reporter.

Since Gaussia luciferase is naturally secreted from mammalian cells in culture (Tannous et al., Mol Ther*11*8, 435-443 (2005), the inventors have discovered that it is secreted into the blood of animals harboring cells expressing this reporter enzyme. Further, expression levels via a constitutive promoter is proportional to cell number irrespective of location of cells in the body, while levels under a physiologic responsive promoter should reflect the physiologic state of the tissue in which the cells were located. Accordingly, the inventors have discovered a method for measuring the level of the naturally secreted Gaussia luciferase in few microliters of biological sample from a subject with cells expressing the Gaussia luciferase, such as blood or urine, as a quantitative index of the number of cells expressing it. The level of Gluc blood in the blood can be used to monitor tumor growth and/or response to a therapy, such as an anti-cancer therapy, as well as the survival and proliferation of circulating cells such as stem cells and T-lymphocytes in vivo. Accordingly in one aspect, the present invention provides a method for a Gluc blood assay which allows a convenient, non-invasive and quantitative assessment of in vivo luciferase reporter activity and should be a significant aid in the monitoring of biological processes in experimental animals.

One aspect of the present invention relates to an assay for identifying an agent which modulates cell growth comprising: (a) measuring the bioluminescent signal from a cell expressing a Gaussia luciferase protein at a first timepoint; (b) contacting the cells with at least one agent; (c) measuring the bioluminescent signal from the cell expressing Gaussia luciferase protein at a second timepoint; wherein a change in the bioluminescent signal measured from the Gaussia luciferase protein at the second time point as compared to the first timepoint identifies an agent which modulates cell growth.

In some embodiments of this aspect and all aspects described herein, modulating is an increase or decrease in cell growth. In some embodiments of this aspect and all aspects described herein, a cell is in a population of cells, and a cell can be any cell, for example but not limited to, a tumor cell.

In some embodiments of this aspect and all aspects described herein a cell is present within a subject or in vivo, in some embodiments, a subject is an animal model of cancer.

In some embodiments of this aspect and all aspects described herein, the bioluminescent signal from the Gaussia luciferase protein is measured in a biological sample obtained from the subject, such as, for example, urine, blood, plasma, lymph fluid, cerebrospinal fluid. In alternative embodiments, the bioluminescent signal from the Gaussia luciferase protein is measured within the subject.

In some embodiments of this aspect and all aspects described herein, the method further comprises a step of measuring the bioluminescent signal from the Gaussia luciferase protein at a second timepoint is performed at several different time points.

In some embodiments of this aspect and all aspects described herein, a cell is contacted with at least one agent is for a sufficient amount of time for modulation of the cell growth to occur.

In some embodiments of this aspect and all aspects described herein, a Gaussia luciferase protein is encoded by the nucleic acid sequence of SEQ ID NO: 1 or a variant or fragment thereof, and in some embodiments, a Gaussia luciferase protein is encoded by a Gaussia luciferase nucleic acid sequence which is codon optimized for mammalian gene expression, such as for example, a humanized Gaussia luciferase (hGLuc) which has the nucleic acid sequence as set forth in SEQ ID NO:3.

In some embodiments of this aspect and all aspects described herein, a cell expressing the Gaussia luciferase protein comprises a nucleic acid construct comprising a nucleic acid encoding the Gaussia luciferase operatively linked to a promoter, such as for example, a tissue specific promoter, or a cancer specific promoter. In alternative embodiments, a promoter is a constitutively active promoter, or an inducible promoter. In some embodiments of this aspect and all aspects described herein, a nucleic acid construct is a vector, for example an expression vector or a viral vector, such as but not limited to, a viral vector selected from a group consisting of: a lentivirus vector, a retroviral vector, a herpes simplex viral vector, an adenovirus vector, an adeno-associated virus (AAV) vector, an EPV vector, an EBV vector and a bacteriophage vector.

Another aspect of the present invention relates to a cell comprising a nucleic acid construct comprising a nucleic acid encoding a Gaussia luciferase protein operatively linked to a promoter.

In some embodiments of this aspect and all aspects described herein, a Gaussia luciferase protein is encoded by the nucleic acid sequence of SEQ ID NO: 1 or a variant or fragment thereof. In some embodiments of this aspect and all aspects described herein, a Gaussia luciferase protein is encoded by a Gaussia luciferase nucleic acid sequence which is codon optimized for mammalian gene expression, for example, a Gaussia luciferase nucleic acid sequence is humanized Gaussia luciferase (hGLuc) and has the nucleic acid sequence as set forth in SEQ ID NO:3.

Another aspect of the present invention relates to an animal comprising a cell comprising a nucleic acid construct comprising a nucleic acid encoding a Gaussia luciferase protein operatively linked to a promoter. In some embodiments of this aspect and all aspects described herein, an animal is a transgenic animal, and in some embodiments, an animal or transgenic animal has been transplanted with the cell comprising a nucleic acid construct comprising a nucleic acid encoding a Gaussia luciferase protein operatively linked to a promoter.

In some embodiments of this aspect and all aspects described herein, an animal is a small experimental animal, for example a rodent.

### BRIEF DESCRIPTION OF FIGURES

FIGS. 1A-1F shows monitoring of Gluc blood levels with subcutaneous tumor model. FIG. 1A shows Different numbers of Gli36-Gluc cells were implanted subcutaneously in mice (n=4) and imaged with a CCD camera three days later. FIG. 1B shows total relative light units (RLU) per second was calculated for tumors in FIG. 1A. Gluc activity was measured in blood (diamonds) or urine (squares) using the luminometer. Results are presented as mean±SD with p<0.001 as calculated by student T-test. FIG. 1C and FIG. 1D shows different numbers of Gli36 cells expressing both Gluc-CFP and SEAP-mCherry, FIG. 1C were implanted subcutaneously in mice and Gluc (in blood) or SEAP (in serum) activity was assayed 2 days later (FIG. 1D). Results are showing as mean±SD with p<0.001. Bar, 100 μm. FIGS. 1E and 1F shows mice were implanted with 1×10⁶ Gli36-Gluc cells subcutaneously and tumor growth was monitored by both in vivo bioluminescence imaging (FIG. 1E) and the Gluc blood assay. In FIG. 1F, at day 10 and 13 post-implantation, one set of mice was injected intra-tumorally (arrows) with an oncolytic HSV vector (10⁸ pfu; red line) and another set with PBS (n=3/group). The results shown are from one representative mouse from each group.

FIGS. 2A-2F shows the Gluc reporter in the blood as a useful tool to monitor in vivo processes. FIGS. 2A and 2B show 1×10⁵ Gli36-Gluc cells were implanted in the brains of nude mice (n=3/group) and tumor growth was monitored by in vivo bioluminescence imaging (FIG. 2A) or by measuring Gluc activity using the luminometer (as shown in FIG. 2B). FIG. 2C and FIG. 2D show 1×10⁶ Gli36 cells were implanted subcutaneously and tumors were either injected with LV-Gluc-CFP or PBS, 3 days later. Viral delivery was monitored over time by measuring Gluc activity in blood samples (as shown in FIG. 2C), by in vivo bioluminescence imaging using the CCD camera (FIG. 2D, upper panel) and by monitoring CFP expression in tumor sections (FIG. 2D, lower panel). FIGS. 2E and 2F show one million C17.2 NPCs expressing Gluc and CFP (FIG. 2E, upper panel) or PBS were injected i.v. in nude mice. Gluc activity was monitored over time using the CCD camera (FIG. 2E, lower panel) and in blood samples using the luminometer (as shown in FIG. 2F). Data shown are from a representative mouse from each set. Scale bar, 100 μm.

FIGS. 3A-3B shows the Gluc blood assay. FIG. 3A shows 20 μl conditioned medium from Gli36-Gluc cells in culture were mixed with 2 μl of either H₂O or 50 mM EDTA and assayed for Gluc activity after adding coelenterazine (100 μM) and acquiring photon counts using a luminometer. EDTA did not show any detrimental effects on Gluc activity. FIG. 3B shows blood samples with EDTA from mice implanted with different amounts of Gli36-Gluc cells were either incubated at 4° C. for 10 min or centrifuged at 2000 g for 10 min to collect the serum and analyzed for Gluc activity. No significant differences in Gluc activity were observed between serum and whole blood under these assay conditions.

FIGS. 4A-4B shows the half-life of Gluc in cell culture medium and blood. FIG. 4A shows conditioned medium from Gli36Gluc cells was incubated at 37° C. and analyzed at different time point for Gluc activity by adding coelenterazine (100 μM) and acquiring photon counts using a luminometer. FIG. 4B shows 200 μl of filtered conditioned medium from Gli36-Gluc cells was injected i.v. in mice after which the Gluc activity in 20 μl blood was measured at different time points after adding coelenterazine (100 μM) and acquiring photon counts using a luminometer. The half-life of Gluc in conditioned medium is about 6 days and in blood about 20 min.

FIGS. 5A-5D show the detection of luciferase in vivo, and corresponding luciferase detected from a biological sample obtained from the mouse or measured in vivo using a CCD camera. FIG. 5A shows different numbers of Gli36-Gluc cells were implanted subcutaneously in mice (n=4) and imaged with a CCD camera three days later. FIG. 5B shows total relative light units (RLU) per second was calculated for tumors in FIG. 5A. Gluc activity was measured in blood using the luminometer or in vivo using a CCD camera. FIG. 5C shows the total relative light units (RLU) per second after days post-implantation of Gluc-expressing cells, demonstrating that as the tumor increases in size, the level of secreted luciferase also increases. FIG. 5D shows an initial decrease in secreted Gluc signal since some of the injected cells lose viability (i.e. die) post-transplantation. After about 24 hrs, the secreted Gluc signal remains relatively constant, indicating that the injected cells are still viable but not proliferating. If the signal decreases further, this indicates that the cells are dying (or losing viability) due to, for example a therapy such as an anti-cancer therapy.

### DETAILED DESCRIPTION

The present invention relates, in part, to the discovery that the secreted forms of luciferases can be used to monitor in vivo (i.e. in an intact cell or a living cell or in a living subject) biological processes in using an non-invasive method comprising measuring the level of a secreted luciferase in an ex vivo biological sample.

As disclosed herein, the inventors have discovered that a nucleic acid encoding a secreted form of luciferase can be administered to a subject, which in some embodiments is useful to monitor disease progression, for example but not limited to cancer progression, in a subject over time by non-invasive procedures.

In some embodiments, the present invention is based, on part, on the discovery that a secreted form of luciferase protein can be used for real-time monitoring of the progression of a disease, such as a cancer in a subject, by measuring the level of bioluminescence present in a biological sample obtained from a subject. In some embodiments, one aspect of the present invention relates to administering to a subject a nucleic acid encoding a secreted luciferase, and in some embodiments, a disease or a diseased tissue, such as the tumor, expresses the secreted luciferase protein. In some embodiments of all aspects described herein, analysis of a secreted form of the luciferase-protein can be monitored in a biological sample obtained from the subject without the need for alternative invasive monitoring procedures. This permits a non-invasive, highly convenient detection, using conventional calorimetric methods and/or assays, of the luciferase protein in biological samples obtained from the subject which comprises the secreted luciferase produced by the tumors at different time points. Therefore, one embodiment is a method for convenient, non-invasive and real-time monitoring of a disease progression, such as the progression of a cancer in a subject using a secreted luciferase protein.

In certain embodiments, repeated monitoring and quantization can be easily performed be by taking repeated biological samples from the subject over time.

Another embodiment of all aspects described herein, as the analysis procedure is non-invasive, multiple biological samples can be obtained and monitored over an extended period of time, for example, at multiple different timepoints, such as daily, weekly, monthly etc, to monitor the efficacy of a disease treatment, for example the efficacy of an anti-cancer agent.

Since the luciferase as used herein is naturally secreted, the methods can be performed on small samples and it makes the methods much faster and more convenient than use of other luciferases such as firefly which is used in the SUPERLIGHT™ luciferase reporter gene assay (Bioassays, CA) where the luciferase is not secreted form the cell into biological samples.

In one embodiment the secreted luciferase protein is expressed within a cell by a nucleic acid construct encoding at least one secreted luciferase gene operatively linked to a regulatory sequence, such as a promoter.

In some embodiments of all aspects described herein, any secreted form of luciferase is useful in the methods and assays as disclosed herein, and includes fragments, variants and recombinant forms of luciferase proteins. In some embodiments, the secreted luciferase is Gaussia luciferase. In another embodiment, the Gaussia luciferase is humanized Gaussia luciferase for expression in mammalian cells. Without being limited to theory, Gaussia luciferase is 2000-fold more sensitive than firefly luciferase (FLuc) and 200-fold than sensitive than alkaline phosphatase (SEAP) in medium. Further, Gaussia luciferase is linear over at-least 10⁷ fold range, whereas SEAP is only linear over a 10⁴ fold range.

In another embodiment the nucleic acid construct is introduced into cells by means of a vector. In another embodiment, the vector is a viral vector. One can use any vector commonly known by one of ordinary skill in the art for use to deliver the nucleic acid construct to a cell. In some embodiments, vectors which can be used include, but are not limited to a lentivirus vector, a retroviral vector, a herpes simplex viral vector, an adenovirus vector, an adeno-associated virus (AAV) vector, an EPV vector, an EBV vector and a bacteriophage.

In one embodiment, one aspect of the present invention relates to the use of the methods and assays described herein for monitoring the effect of an agent on a population of cells, such as an anti-cancer agent to decrease a population of cancer or tumor cells. In such an embodiment, the methods of the invention described herein are advantageous over previous efficacy methods to determine the effect of an agent on reducing at least one symptom of a cancer, in that the methods as disclosed herein enable a real time, quick and non-invasive in vivo method to repeatedly monitor the level of secreted luciferase in a biological sample at multiple time points from the subject, which directly correlates with the tumor size, and thus provides a reliable indicator of the effect of the anti-cancer agent at reducing the tumor size or attenuating the rate of tumor growth in the subject over the multiple time points when the biological samples were taken from the subject. Thus, the methods as disclosed herein have an advantage of analyzing the efficacy of a therapeutic agent, such as an anti-cancer agent by monitoring the level of the secreted luciferase protein in a biological sample from the subject at distinct time points during the course of an anti-cancer treatment, for example at the start of therapy (i.e. at a first timepoint) and at subsequent timepoints (i.e. a second, third, fourth, fifth and so forth) timepoints following the first timepoint and initiation of treatment. In some embodiments, one can compare the level of the secreted luciferase protein in a biological sample from the subject taken at any combination of time points (i.e. 2^{nd} v. 3^{rd} timepoint, or 2^{nd} vs. 4^{th} time point or 1^{st} vs. 3^{rd} timepoint, or 1^{st} v. 2^{nd}, 3^{rd}, 4^{th} etc. timepoints) where if the level of secreted luciferase has decreased from an later timepoint as compared to an earlier timepoint, then it indicates a decrease in the number of luciferase expressing cells. Accordingly, the methods as disclosed herein provide a sensitive assay to analyze the disease progression in vivo, such as tumor size in a subject using non-invasive procedures.

In some embodiments, a secreted luciferase protein as disclosed herein comprises Gaussia luciferase (GLuc) and has the nucleic acid sequence as set forth in SEQ ID NO:1. In some embodiments, the nucleic acid sequence for Gaussia luciferase (GLuc) is codon optimized for mammalian gene expression, for example for expression in human cells, for example the Gaussia luciferase is humanized is humanized Gaussia luciferase (hGLuc) and has the nucleic acid sequence as set forth in SEQ ID NO.3.

In some embodiments, the nucleic acid construct as disclosed herein is present or is a vector, for example an expression vector. In alternative embodiments, the vector is a viral vector, for example, but not limited to viral vector such as, a lentivirus vector, retroviral vector, lentivirus vector, herpes simplex viral vector, adenovirus vector, adeno-associated virus (AAV) vectors, EPV, EBV or variants or derivatives thereof.

In some embodiments, methods to measure bioluminescence are commonly known by persons of ordinary skill in the art and include, for example but not limited to measuring the bioluminescence by detecting luciferase using a microplate luminometer or using a CCD imaging system. In some embodiments, the methods as disclosed herein further comprise measuring an additional signal from the cell, wherein the additional signal measures the signal from the marker gene to identify the cells containing the vector.

Another aspect of the present invention relates to a vector comprising the nucleic acid construct as disclosed herein, such as a vector comprising a nucleic acid encoding a secreted luciferase operatively linked to a regulatory sequence. Another aspect of the present invention relates to a cell comprising such a vector, for example a mammalian cell. In some embodiments, the cell is in vivo, and in alternative embodiments, the cell is in vitro or ex vivo. In some embodiments, the cell is a human cell.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, chemiluminescence refers to a chemical reaction in which energy is specifically channeled to a molecule causing it to become electronically excited and subsequently to release a photon thereby emitting visible light. Temperature does not contribute to this channeled energy. Thus, chemiluminescence involves the direct conversion of chemical energy to light energy.

As used herein, luminescence refers to the detectable EM radiation, generally, UV, IR or visible EM radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules [or synthetic versions or analogs thereof] as substrates and/or enzymes.

The term “bioluminescence” as used herein is a type of chemiluminescence, refers to the emission of light by biological molecules, particularly protons. Essential conditions for bioluminescence comprise; molecular oxygen, either bound or free in the presence of an oxygenase; a luciferase, which acts on a substrate luciferin. The bioluminescence reaction is an energy-yielding chemical reaction in which a specific chemical substrate, a luficerin, undergoes oxidation, catalyzed by an enzyme, a luciferase.

The term “luciferase” refers to enzymes that catalyze the oxidation of luciferin, emitting light and releasing oxyluciferin. The term “bioluminescence-system” refers to the components that are necessary and sufficient to generate bioluminescence. These include luciferase, luciferins and any necessary co-factors or conditions. Virtually any bioluminescent system known to those skilled in the art will be amenable to use in the methods described herein.

The term “bioluminescence substrate” as used herein refers to the compound that is oxidized in the presence of luciferase and any necessary activators, and generates light. These substrates are referred to “luciferins” herein, and are substrates that undergo oxidation in a bioluminescence reaction. These bioluminescence substrates include any luciferin or analogue thereof, or any synthetic compound that generates light. Such molecules include naturally-occurring substrates, modified forms thereof and synthetic substrates [see e.g. U.S. Pat. Nos. those described in U.S. Pat. Nos. 5,374,534 and 5,098,828 which are incorporated herein in their entirety by reference]. Exemplary luciferins include those described in U.S. Pat. No. 6,436,682, and derivatives thereof, analogues thereof, synthetic substrates, as well as dioxetanes [see e.g. U.S. Pat. Nos. 5,004,565 and 5,455,357 which are incorporated herein in their entirety by reference], and other compounds that are oxidized by luciferase in a light-producing reaction [see, e.g. U.S. Pat. Nos. 5,374,534, 5,098,828 and 4,950,588 which are incorporated herein in their entirety by reference]. Such substrates may be identified empirically by selecting compounds that are oxidized in bioluminescent reactions. Bioluminescence substrates, thus, include those compounds that those skilled in the art recognize as luficerins. In one embodiment, the luciferin is coelenterazine and analogues thereof, which include molecules in U.S. Pat. No. 6,436,682, which is incorporated herein in its entirety by reference, and for example, see Zhao et al, (2004), Mol Imaging, 3; 43-54.

Bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein [luciferase] that is an oxygenase that acts on a substrate luciferin [a bioluminescence substrate] in the presence of molecular oxygen and transforms the substrate to an excited state, which upon return to a lower energy level releases the energy in the form of light.

Methods to measure bioluminescence are well known to those skilled in the art. Bioluminescence reactions are also well-known to those skilled in the art, and any such reaction may be adapted for used in combination with articles of manufacture as described herein. In another embodiment, bioluminescence can be measured by Bioluminescence Resonance Energy Transfer (BRET) (Boute et al, (2002) Trends. Pharmacol. Sci. 23; 351-354; Morin & Hastings (1971) J Physioll 77; 313-18) refers to the natural phenomena whereby green bioluminescence emission observed in vivo was shown to be the result of the luciferase non-radioactively transferring energy to an accessory green fluorescent protein (GFP). Energy transfer between two flurescent proteins (FRET) as a physiological reporter has been reported (Miyawaki et al, 1997, Natuew, 388; 882-7). A similar reported is possible with a luciferase-GFP pair (see, e.g. U.S. Pat. No. 6,436,682 which is incorporated in its entirety herein for reference).

As used herein, the substrates and enzymes for producing bioluminescence are generically referred to as luciferin and luciferase, respectively. When reference is made to a particular species thereof, for clarity, each generic term is used with the name of the organism from which it derives, for example, bacterial luciferin or firefly luciferase.

As used herein, luciferase refers to oxygenases that catalyze a light emitting reaction. For instance, bacterial luciferases catalyze the oxidation of flavin mononucleotide [FMN] and aliphatic aldehydes, which reaction produces light. Another class of luciferases, found among marine arthropods, catalyzes the oxidation of Cypridina [Vargula] luciferin, and another class of luciferases catalyzes the oxidation of Coleoptera luciferin.

Thus, luciferase refers to an enzyme or photoprotein that catalyzes a bioluminescent reaction [a reaction that produces bioluminescence]. The luciferases, such as firefly and Gaussia and Renilla luciferases, that are enzymes which act catalytically and are unchanged during the bioluminescence generating reaction. The luciferase photoproteins, such as the aequorin photoprotein to which luciferin is non-covalently bound, are changed, such as by release of the luciferin, during bioluminescence generating reaction. The luciferase is a protein that occurs naturally in an organism or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

The term “luciferase” or “luciferases” used interchangeably herein, refers to an enzyme or photoprotein that catalyzes a bioluminescent reaction [a reaction that produces bioluminescence]. Luciferases are catalytically and are unchanged during bioluminescence. Luciferase photoproteins to which luciferin is non-covalently bound, are changed, such as by the release of luciferin during the bioluminescence reaction. “Luciferin” used to herein, refers any compound that, in the presence of any necessary activators, catalyze the oxidation of a bioluminescence substrate in the presence of molecular oxygen, whether free or bound, from a lower energy state to a higher energy state, such that the substrate upon return to the lower energy state, emits light.

“Gauissa Luciferase” used interchangeably as “Gluc” or “GLuc” herein, refers to the luciferase enzyme isolated from member of the genus Gaussia or an equivalent molecule obtained from any other source, such as from another related copepod, or has been prepared synthetically. It is intended to encompass Gaussia luciferase with conservative amino acid substitutions that do not substantially alter activity. Suitable conservative substitutions of amino acids are known to those skilled in the art and may be made generally without altering the biological activity of the resulting molecule. Those of skill in the art, recognize that, in general, single amino acid substitutions in non-essential regions of the polypeptide do not alter biological activity (see e.g. Watson et al, Molecular biology of the gene, 4^{th} Ed, 1987, p 224)

The DNA encoding Gaussia Luciferase is available commercially, for example ProLume Ltd and Nanotlight. Alternatively, methods of isolating the DNA encoding Gaussia luciferase from natural sources are described in U.S. Pat. No. 6,436,682 and are incorporated herein for reference. Methods of producing mutants containing a predetermined nucleotide sequence are well known in the art. Two widely known methods are Kunkel mutagenesis and PCR mutagenesis. A detailed description can be found in Current Protocols in Molecular Biology, Wiley Interscience, 1987, sections 8.1 and 8.5 respectively. Kunkel mutagenesis is also described in an article by Kunkel in Proc. Acad. Natl. Sci, USA, 82; 488-492, while PCR is discussed in an article by Saiki et al, Science 239, 487-491.

The term “humanized Gaussia luciferase” or “hGluc” are used interchangeably, and refer to a humanized form of a Gauissa Luciferase nucleic acid sequence, in which the nucleic acid sequence has been modified for expression in mammalian cells, and correspond to SEQ ID NO:3. A detailed description for the methods for producing humanized Gaussia luciferase can be found in Tannous et al, (2005) Mol Therapy, 11; 435-443.

The luciferases and luciferin and activators thereof are referred to as bioluminescence generating reagents or components. Typically, a subset of these reagents will be provided or combined with an article of manufacture. Bioluminescence will be produced upon contacting the combination with the remaining reagents. Thus, as used herein, the component luciferases, luciferins, and other factors, such as O2, Mg2+, Ca2+ are also referred to as bioluminescence generating reagents [or agents or components].

As used herein, bioluminescence substrate refers to the compound that is oxidized in the presence of a luciferase, and any necessary activators, and generates light. These substrates are referred to as luciferins herein, are substrates that undergo oxidation in a bioluminescence reaction. These bioluminescence substrates include any luciferin or analog thereof or any synthetic compound with which a luciferase interacts to generate light. Preferred substrates are those that are oxidized in the presence of a luciferase or protein in a light-generating reaction. Bioluminescence substrates, thus, include those compounds that those of skill in the art recognize as luciferins. Luciferins, for example, include firefly luciferin, Cypridina [also known as Vargula] luciferin [coelenterazine], bacterial luciferin, as well as synthetic analogs of these substrates or other compounds that are oxidized in the presence of a luciferase in a reaction the produces bioluminescence.

As used herein the term “secreted luciferase” refers to a luciferase that, in its native form, is secreted from the cell in which it is normally expressed into an extracellular location. The extracellular location may be internal or external to the organism depending on the identity of the organism. The extracellular location includes within its scope the medium in which a cell expressing the luciferase is being cultured in vitro. It may also include the aquatic or marine environment in which the organism that expresses the luciferase usually resides. It should also be noted that encompassed by the term “secreted luciferase” are various modified and recombinant forms thereof as described herein, wherein such recombinant or modified forms may or may not be secreted. Thus, the term “secreted luciferase” includes within its scope “non-secreted” forms of secreted luciferases.

As used herein the term “non-secreted luciferase” means a luciferase that is not exported or secreted from a cell into the extracellular environment. Thus “non-secreted” includes a luciferase retained in the cell in any form, and thus the luciferase may be cytoplasmic or membrane-associated. Typically, although not exclusively, where a luciferase is referred to herein as being a “non-secreted” form of a luciferase that is secreted in its native form, this secretion and absence of secretion refers to eukaryotic cells.

As used herein the term “intracellular luciferase” refers to a luciferase that upon expression, and in its native form, is retained within the cell or cell membrane rather than secreted into the extracellular medium. It should be noted that encompassed by the term “intracellular luciferase” are various modified and recombinant forms thereof as described herein, wherein such recombinant or modified forms may or may not remain intracellular. Thus, the term “intracellular luciferase” includes within its scope exported or secreted forms of intracellular luciferases. Thus, as used herein the terms “non-secreted” and “intracellular” have distinct meanings. A “non-secreted” luciferase may be one that is a modified form of a luciferase that is secreted in its native form, whereas an “intracellular” luciferase is one that is intracellular (not secreted) in its native form.

As used herein the terms “inactivate”, “inactivation” and variations thereof as used herein mean, in the context of inactivation of a luciferase by a reducing agent, that the catalytic activity of the luciferase as determined by the amount of luminescent signal generated is reduced or abolished. Thus, inactivation may be complete in that 100% reduction in activity may result, but this need not be the case. Partial inactivation such that some residual activity is retained is also contemplated. As used herein with reference to luciferase enzymes, the term “substrate” means the reactive substrate molecule upon which the luciferase acts, excluding any additional cofactors that may be beneficial to, or required for, binding of the luciferase to the substrate and/or catalysis. For example, luciferase catalysed reactions may require or benefit from cofactors such as magnesium, CoA and ATP, however in the context of the present invention such cofactors are not considered to fall within the scope of the term “substrate”. Luciferase “substrates” include for example D-luciferin and coelenterazine. For the purposes of the present application the term “luciferin” refers to the substrate D-luciferin and its analogues, which molecules are substrates for luciferases derived from, for example, Coleoptera, such as firefly, click beetles and railroad worms. In the context of the present invention the term luciferin does not encompass coelenterazine, which represents a different luciferase substrate utilized by a distinct class of luciferase (such as those derived from Renilla, Gaussia and Metridia for example).

As used herein, capable of conversion into a bioluminescence substrate means susceptible to chemical reaction, such as oxidation or reduction, that yields a bioluminescence substrate. For example, the luminescence producing reaction of bioluminescent bacteria involves the reduction of a flavin mononucleotide group (FMN) to reduced flavin mononucleotide (FMNH2) by a flavin reductase enzyme. The reduced flavin mononucleotide [substrate] then reacts with oxygen [an activator] and bacterial luciferase to form an intermediate peroxy flavin that undergoes further reaction, in the presence of a long-chain aldehyde, to generate light. With respect to this reaction, the reduced flavin and the long chain aldehyde are substrates.

As used herein, a bioluminescence generating system refers to the set of reagents required to conduct a bioluminescent reaction. Thus, the specific luciferase, luciferin and other substrates, solvents and other reagents that may be required to complete a bioluminescent reaction form a bioluminescence system. Thus a bioluminescence generating system refers to any set of reagents that, under appropriate reaction conditions, yield bioluminescence. Appropriate reaction conditions refers to the conditions necessary for a bioluminescence reaction to occur, such as pH, salt concentrations and temperature. In general, bioluminescence systems include a bioluminescence substrate, luciferin, a luciferase, which includes enzymes luciferases and photoproteins, and one or more activators. A specific bioluminescence system may be identified by reference to the specific organism from which the luciferase derives; for example, the Vargula [also called Cypridina] bioluminescence system (or Vargula system) includes a Vargula luciferase, such as a luciferase isolated from the ostracod, Vargula or produced using recombinant means or modifications of these luciferases. This system would also include the particular activators necessary to complete the bioluminescence reaction, such as oxygen and a substrate with which the luciferase reacts in the presence of the oxygen to produce light.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, an optional operate sequence to control transcription, a sequence encoding suitable mRNA ribosomal binding sites, and sequences to control the termination of transcription and/or translation. Regulatory sequences are selected for the assay to control the expression of Gaussia Luciferase-fluorescent fusion protein in a cell-type in which expression is intended.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

As used herein, a “promoter” or “promoter region” or “promoter element” used interchangeably herein, refers to a segment of DNA or RNA that controls transcription of the DNA or RNA to which it is operatively linked. Typically, it refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated. Promoters, depending upon the nature of the regulation, may be constitutive or regulated. Exemplary promoters contemplated for use in prokaryotes include the bacteriophage T7 and T3 promoters, and the like.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like.

The term “operatively linked” or “operatively associated” are used interchangeably herein, and refer to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The term “IRES” refers to internal ribosome entry sites (see Kozak (1991) J. Biol. Chem. 266'19867-70) are sequences encoding consensus ribosome binding sites, and can be inserted immediately 5′ of the start codon and/or regulatory sequences or elsewhere 5′ of nucleic acid sequences encoding genes or marker genes to enhance expression of the downstream nucleic acid sequence. The desirably of, or need for, such modification may be empirically determined.

The term “marker gene” refers to any gene whose expression can be detected. Many such genes are known in the art. One or more marker genes are useful for the identification and/or selection of host cells which contain the nucleic acid construct as disclosed herein. Cells can be screened for a marker present in the construct. Various markers include hprt, resistance genes such as neomycin resistance, thymidine kinase, hygromycin resistance etc, and various cell-surface markers such as Tac, CD8, CD3, thyl, NGF receptor etc., as well as fluorescent proteins, which are well known in the art and include without limitation, GFP, CRP, RFP, and the like.

The term “fluorescent protein” refers to a protein that possesses the ability to fluoresce (i.e., to absorb energy at one wavelength and emit it at another wavelength). For example, a green florescent protein refers to a polypeptide that has a peak in the emission spectrum at about 510 nm. These proteins can be used as a fluorescent label or marker and in any application in which such labels would be used, such as immunoassays, CRET, FRET, and FET assays, and in the assays such as the BRET (Bioluminescence Resonance Energy Transfer) in U.S. Pat. No. 6,436,682 which refers to any method in which luciferase is used to generate light upon reaction with luciferin which is non-radioactively transferred to a fluorescent protein.

The term “cell” used herein refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells in this invention.

Cells also refer not to a particular subject cell but to the progeny or potential progeny of such a cell because of certain modifications or environmental influences, for example differentiation, such that the progeny mat not, in fact be identical to the parent cell, but are still included in the scope of the invention. Preferably cells of the present invention are in vivo. In some embodiments, the cells can be ex vivo or cultured cells, e.g. in vitro. For example, for ex vivo cells, cells can be obtained from a subject, where the subject is healthy and/or affected with a disease. Cells can be obtained, as a non-limiting example, by biopsy or other surgical means know to those skilled in the art. Accordingly, cells used in the methods as disclosed herein can present in a subject, e.g. as part of an in vivo assay. In some embodiments, the methods as disclosed herein can be performed on in vivo cells, where the cell is preferably found in a subject. Methods for assaying bioluminescence are described in Tannous et al, (2005) Mol Therapy, 11; 435-443 and other methods known to persons skilled in the art.

The terms “patient”, “subject” and “individual” are used interchangeably herein, and refer to an animal, particularly a human, to whom administration of the nucleic acid construct as disclosed herein is provided. As used herein, the term “subject” is intended to include human and non-human animals. The term “non-human animals” includes all vertebrates, e.g. mammals, non-mammals, such as non-human primates, sheep, dog, cow, chickens, amphibians, reptiles etc. In certain embodiments, the subject is mammal, e.g., a primate, e.g., a human. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

As used herein the term “sample” means any sample, including but not limited to cells, organisms, lysed cells, extracts or components of cells or organisms, extracellular fluid, and media in which cells are cultured, wherein the sample contains or is suspected to contain a luciferase(s) enzyme.

In order for a particular cell to express the proteins encoded by nucleic acid sequences, the nucleic acid must be introduced into the cell. Methods to introduce DNA into cells, the cell must be transformed by an appropriate vector. “Transformation”, as used herein, refers to the introduction of heterologous polynucleotide or nucleic acid sequence or fragment thereof into a host cell, irrespective of the method used, for example direct uptake, transfection or transduction. The present invention, therefore also relates to cells which have been transformed by at least one nucleic acid construct, wherein one construct comprises the sequence for Gaussia luciferase fluorescent conjugate protein of the present invention and expresses the Gaussia luciferase-fluorescent conjugate protein. The construct may be introduced into the cell by multiple means known to persons skilled in the art, including vectors, viral vectors, and non-viral means. Non-viral means include without limitation, fusion, electroporation, biolistics, transfection, lipofection, protoplast fusion, calcium phosphate transfection, microinjection, pressure-forced entry, naked DNA etc. or any other means known any person skilled in the art.

The term “vectors” used interchangeably with “plasmid” refer to a nucleic acid molecule capable of transporting another nucleic acid to which it has been linked. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

In one embodiment, the expression vector used herein are replicable DNA constructs which the nucleic acid is operatively linked to a suitable regulatory sequence capable of affecting the expression of the Gaussia luciferase-fluorescent protein conjugate. The construct may be introduced by homologous recombination, where it is desired that a construct be integrated at a particular locus. For example, one can delete and/or replace an endogenous gene at the same locus or elsewhere with the nucleic acid construct of this invention. For homologous recombination, the nucleic acid is cloned into specific vectors, including but not limited to; Ω or O-vectors, see, for example, Thomas and Capecchi, cell, (1987), 51; 503-512, Mansour et al, nature, (1988) 336; 348-352; and Joyner et al, nature (1989) 338; 153-156.

Vectors comprising useful elements such as bacterial or yeast origins of replication, selectable and/amplifiable markers, promoter/enhancer elements for the expression in prokaryotes or eukaryotes, and mammalian expression control elements etc, may be used to prepare stocks of nucleic acid constructs and for carrying out transfections are well known in the art, and many are commercially available.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

Methods to induce a fluorescence signal require the excitation of the fluorescent protein at a particular wavelength to cause fluorescence excitation of the particular fluorescent protein. Detection of this fluorescence signal required. Methods to measure fluorescence may be carried out for example by fluorimetry, FACS and by microscope techniques well known by one skilled in the art. In this manner localization and/or quantification of the fluorescent protein may be determined.

The term “agent” as used herein refers to any entity which is normally not present or not present at the levels being administered in the cell. Agents may be selected from a group comprising; chemicals; an action; nucleic acid sequences; proteins; peptides; or fragments thereof. An agent can be a nucleic acid sequence, such as RNA or DNA, and may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. An agent can also be a protein and/or peptide or fragment thereof, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. An agent also includes to any action and/or event the cells are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, fluorescence exposure etc. Environmental stimuli also include intrinsic environmental stimuli defined below. The exposure to environmental stimuli may be continuous or non-continuous.

The term “compound of interest” refers to any chemical, entity or moiety, including without limitation synthetic and naturally-occurring non-proteinaceous entities. In certain embodiments the compound of interest is a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “nucleic acid” or “oligonucleotide” or “polynucleotide” used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphophoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O— and N— alkylated nucleotides, e.g. N6-methyl adenosine are suitable. The 2′ OH-group can be replaced by a group selected from H, OR, R, halo, SH, SR, NH₂, NHR, NR₂ or CN, wherein R is C—C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

As used herein, to target a targeted agent, such as a luciferase, means to direct it to a cell that expresses a selected receptor or other cell surface protein by linking the agent to a such agent. Upon binding to or interaction with the receptor or cell surface protein the targeted agent, can be reacted with an appropriate substrate and activating agents, whereby bioluminescent light is produced and the tumorous tissue or cells distinguished from non-tumorous tissue.

As used herein, an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, visualizable means detectable by eye, particularly during surgery under normal surgical conditions, or, if necessary, slightly dimmed light.

As used herein, pharmaceutically acceptable salts, esters or other derivatives of the conjugates include any salts, esters or derivatives that may be readily prepared by those of skill in this art using known methods for such derivatization and that produce compounds that may be administered to animals or humans without substantial toxic effects and that either are pharmaceutically active or are prodrugs.

As used herein, treatment means any manner in which the symptoms of a conditions, disorder or disease are ameliorated or otherwise beneficially altered. Treatment also encompasses any pharmaceutical use of the compositions herein. Preferably, the term “treating” includes reducing or alleviating at least one adverse effect or symptom of a condition, disease or disorder associated with cancer. As used herein, the term treating is used to refer to the reduction of a symptom and/or a biochemical marker of cancer by at least 10%. For example but are not limited to, about a 10% or more reduction in a the size of the tumor, or about a 10% or more reduction in the rate of growth of the tumor or about a 10% or greater decrease in size of the tumor growth of the tumor would also be considered as affective treatments by the methods as disclosed herein.

The term “effective amount” as used herein refers to the amount of therapeutic agent of pharmaceutical composition to reduce or stop at least one symptom of the disease or disorder, for example a symptom or disorder of a tumor. For example, an effective amount using the methods as disclosed herein would be considered as the amount sufficient to reduce a symptom of the disease or disorder, for example to reduce the progression of the cancer by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease.

As used herein, an effective amount in reference to a nucleic acid encoding a Gauissa luciferase protein for diagnosing a disease is an amount that will result in a detectable tissue. The tissues are detected by visualization either without aid from a detector more sensitive than the human eye, or with the use of a light source to excite any fluorescent products.

As used herein, amelioration of the symptoms of a particular disorder by administration of a particular pharmaceutical composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities may be observed in in vitro systems designed to test or use such activities. Thus, for purposes herein the biological activity of a luciferase is its oxygenase activity whereby, upon oxidation of a substrate, light is produced.

As used herein, targeting agent refers to an agent that specifically or preferentially targets a linked targeted agent, a luciferin or luciferase, to a neoplastic cell or tissue.

As used herein, tumor antigen refers to a cell surface protein expressed or located on the surface of tumor cells.

As used herein, neoplastic cells include any type of transformed or altered cell that exhibits characteristics typical of transformed cells, such as a lack of contact inhibition and the acquisition of tumor-specific antigens. Such cells include, but are not limited to leukemic cells and cells derived from a tumor.

As used herein, neoplastic disease is any disease in which neoplastic cells are present in the individual afflicted with the disease. Such diseases include, any disease characterized as cancer.

As used herein, metastatic tumors refers to tumors that are not localized in one site.

As used herein, specialty tissue refers to non-tumorous tissue for which information regarding location is desired. Such tissues include, for example, endometriotic tissue, ectopic pregnancies, tissues associated with certain disorders and myopathies or pathologies.

As used herein, the terms “administering,” and “introducing” are used interchangeably and refer to the placement of the nucleic acid construct as disclosed herein into a subject by a method or route which results in at least partial localization of the agents at a desired site. The compounds of the present invention can be administered by any appropriate route which results in an effective treatment in the subject.

The articles “a” and “an” are used herein to refer to one or to more than one (i.e., to at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the following examples which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### General Methods of the Assay

As disclosed herein, in one embodiment, the method of the present invention involves the introduction into a cell which is present in a subject, for example a human, of a nucleic acid construct encoding a secretable form of a luciferase enzyme which is operatively linked to a regulatory sequence. In one embodiment, the luciferase is from the Gaussia genus. In another embodiment, Gaussia luciferase protein is used. The introduction of the nucleic acid can be by any method described above.

In some embodiments, the nucleic acid construct as disclosed herein is introduced into cells from any species and any tissue. In some embodiment, the cells are present in a subject (i.e. the cells are in vivo). In alternative embodiments, the cells are ex vivo, for example the cells can be removed from the subject and optionally cultured or maintained in a conventional culture medium under suitable conditions permitting growth of the cells, and re-introduced into the subject. For example, the cell is cultured in standard tissue culture media containing the necessary reagents to select for cells which stably retain the nucleic acid construct described above. Cells may be cultured in standard tissue culture dishes e.g. multidishes and microwell plates, or in other vessels, as desired. In some configurations, the assay can be conducted in a 96 well; 386-well or other multi-well plates.

The nucleic acid constructs as disclosed herein comprising a nucleic acid encoding a Gaussia luciferase protein operatively linked to the regulatory sequence, can be introduced simultaneously, or consecutively, each with the same or different markers.

One aspect of the present invention relies on the fact that the cells with the introduced nucleic acid constructs as disclosed herein are capable of expressing the secreted luciferase protein; comprising steps of gene transcription, translation and post-translational modification. The expressed secreted luciferase protein is secreted out of the cell, and in some embodiments into biological samples such as the blood. In some embodiments, the kidney filters the secreted luciferase protein from the blood and it is expelled from the body in waste biological samples, such as urine biological samples.

The expression of the secreted luciferase protein present in biological samples from the subject can be assessed by bioluminescence. In one embodiment, bioluminescence is used as a measure of expression of the secreted luciferase protein in the transfected cells, and its subsequent secretion into the subjects biological samples. In one embodiment, a luciferin, for example but not limited to, coelenterazine and its analogues is added to the biological sample from the subject, and the bioluminescence monitored. In another embodiment, biological samples are collected at different time intervals, and optionally stored at +4° C. or −20° C. for a period of time and the subsequently assayed for bioluminescence. In such an embodiment, biological samples can be obtained from the subject at multiple time points, for example during a particular treatment regimen, and assessed for luciferase activity as in indicator to the disease progression or effectiveness of the treatment regime.

In some embodiments, further analysis and monitoring of the disease progression in the subject can be done by taking biological samples from the subject at specific time points in a treatment regimen, for example, before treatment, during treatment and after treatment, for example to monitor tumor regression during treatment and/or to monitor for re-appearance of tumor cells.

Since the Gaussia luciferase is naturally secreted, the assay can be performed on small samples of the biological sample, such as but not limited to, blood or urine samples, without the need for invasive monitoring systems, and/or expensive monitoring apparatus, such as MRI or other visualization methods to detect the size of tumors in subjects. Accordingly, the present invention enables easy monitoring of the disease progression and/or regression of a tumor using non-invasive, quick methods that can be carried out in the subjects local medical facility without the need for specialist equipment.

In some embodiments, a biological sample can be obtained from a subject at one location and transferred to another location where the biological sample is measured for the level of bioluminescence. By way of an example only, a subject which has been administered the nucleic acid constructs as disclosed herein can obtain a biological sample, such as a blood sample or urine sample, either while attending a medical facility or not at a medical facility, and send the biological sample to a different facility, where the level of bioluminescence in the biological sample is measured. In some embodiments, a physician reviewed the results of the level of the bioluminescence present in the biological sample from one time point to another timepoint, and depending on the results recommends the subject to be treated accordingly. For example, if the subject is undergoing a treatment with an anti-cancer agent and physician determines there is a decrease in the level of bioluminescence in the biological sample from the second timepoint as compared to the first timepoint, the physician may recommend maintenance of the anti-cancer agent until the bioluminescence signal is almost or preferably completely eliminated from the biological sample at a subsequent timepoint. Alternatively, in another sinario, if the subject is undergoing treatment with an anti-cancer agent and physician determines there is an increase in the level of bioluminescence in the biological sample from the second timepoint as compared to the first timepoint, the physician may recommend an increased dose of the anti-cancer agent or an alternative therapy, such as a different anti-cancer agent or therapy, or a combination of anti-cancer therapies. Accordingly, in such embodiments, the subject will continue to be monitored for progression of the cancer at subsequent time points until the bioluminescence signal is almost or preferable completely eliminated from the biological sample at a subsequent timepoint.

Accordingly, the present invention provides methods and nucleic acid constructs for a much faster, non-invasive, easy and highly convenient way to monitor progression of a disease such as cancer in a subject. The present invention is advantageous over other systems in that the methods do not require expensive equipment or apparatus, such as MRI machines to routinely monitor the progression of a disease in a subject, such as cancer.

### Delivery of the Nucleic Acid Constructs to the Cells

In one aspect of the present invention, the nucleic acid constructs as disclosed herein are administered to a subject in any means commonly known by person of ordinary skill in the art. In some embodiments, the nucleic acid constructs as disclosed herein are delivered in a pharmaceutically acceptable carrier. Additional carrier agents, such as liposomes, can be added to the pharmaceutically acceptable carrier.

In another embodiment, the nucleic acid constructs can be delivered by a vector encoding the nucleic acid constructs as disclosed herein, where the vector is a pharmaceutically acceptable carrier, and the composition comprising the vector encoding the nucleic acid construct as disclosed herein is administered to the cells in a subject, such as tumor cells or cells of an organ which is cancerous or comprises tumor lesions.

In one embodiment, the nucleic acid constructs can be delivered by a vector can be taken up actively by cells in vivo following intravenous injection, e.g., hydrodynamic injection. In some embodiments, the nucleic acid constructs can be delivered without the use of a vector, such as delivery of naked nucleic acid by methods commonly known by person of ordinary skill in the art.

Other strategies for delivery of the nucleic acid constructs as disclosed herein can be by delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, nucleic acid constructs as disclosed herein can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. Inducible promoters are well known in the art, and include for example, but are not limited to; tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like. Inducible promoters are characterized by resulting in additional transcription activity when in the presence of, influenced by, or contacted by the inducer than when not in the presence of, under the influence of, or in contact with the promoter. The inducer may be endogenous, or a normally exogenous compound or protein that is administered in such a way as to be active in inducing expression from the inducible promoter. In some embodiments, the inducer agent, i.e. a compound or protein, can itself be the result of transcription or expression of a polynucleotide (i.e. can be a repressor protein), which itself may be under the control or an inducible or repressible promoter.

Inducible promoters useful in the methods and systems of the present invention are capable of functioning in a eukaryotic host organism. Preferred embodiments include mammalian inducible promoters, although inducible promoters from other organisms as well as synthetic promoters designed to function in a eukaryotic host may be used. The important functional characteristic of the inducible promoters of the present invention is their ultimate inducibility by exposure to an externally applied agent, such as an environmental inducing agent. Appropriate environmental inducing agents include exposure to heat, various steroidal compounds, divalent cations (including Cu⁺² and Zn⁺²), galactose, tetracycline, IPTG (isopropyl-β-D thiogalactoside), as well as other naturally occurring and synthetic inducing agents and gratuitous inducers.

The nucleic acid construct and systems disclosed herein encompass the inducibility of a eukaryotic promoter by either of two mechanisms. In particular embodiments of the present invention, the nucleic acid construct comprises suitable inducible promoters can be dependent upon transcriptional activators that, in turn, are reliant upon an environmental inducing agent. In some embodiments, the inducible promoters can be repressed by a transcriptional repressor which itself is rendered inactive by an environmental inducing agent. Thus the inducible promoter can be either one that is induced by an environmental agent that positively activates a transcriptional activator, or one which is derepressed by an environmental agent which negatively regulates a transcriptional repressor. For example, as demonstrated in the Examples, one inducible promoter used is the LacO system, whereby addition of the externally added agent IPTG negatively regulates the transcriptional repressor LacI.

Inducible promoters useful in the methods and systems as disclosed herein include those controlled by the action of latent transcriptional activators that are subject to induction by the action of environmental inducing agents. Preferred examples include the copper-inducible promoters of the yeast genes CUP1, CRS5, and SOD1 that are subject to copper-dependent activation by the yeast ACE1 transcriptional activator (see e.g. Strain and Culotta, 1996; Hottiger et al., 1994; Lapinskas et al., 1993; and Gralla et al., 1991). Alternatively, the copper inducible promoter of the yeast gene CTT1 (encoding cytosolic catalase T), which operates independently of the ACE1 transcriptional activator (Lapinskas et al., 1993), can be utilized. The copper concentrations required for effective induction of these genes are suitably low so as to be tolerated by most cell systems, including yeast and Drosophila cells. Alternatively, other naturally occurring inducible promoters can be used in the present invention including: steroid inducible gene promoters (see e.g. Oligino et al. (1998) Gene Ther. 5: 491-6); galactose inducible promoters from yeast (see e.g. Johnston (1987) Microbiol Rev 51: 458-76; Ruzzi et al. (1987) Mol Cell Biol 7: 991-7); and various heat shock gene promoters. Many eukaryotic transcriptional activators have been shown to function in a broad range of eukaryotic host cells, and so, for example, many of the inducible promoters identified in yeast can be adapted for use in a mammalian host cell as well. For example, a unique synthetic transcriptional induction system for mammalian cells has been developed based upon a GAL4-estrogen receptor fusion protein that induces mammalian promoters containing GAL4 binding sites (Braselmann et al. (1993) Proc Natl Acad Sci USA 90: 1657-61). These and other inducible promoters responsive to transcriptional activators that are dependent upon specific inducing agents are suitable for use with the present invention.

An inducible promoter useful in the methods and systems as disclosed herein can be induced by one or more physiological conditions, such as changes in pH, temperature, radiation, osmotic pressure, saline gradients, cell surface binding and the concentration of one or more extrinsic or intrinsic agents. The extrinsic agent may comprise amino acids and amino acid analogs, saccharides and polysaccharides, nucleic acids, transcriptional activators and repressors, cytokines, toxins, petroleum-based compounds, metal containing compounds, salts, ions, enzyme substrate analogs and combinations thereof. In specific embodiments, the inducible promoter is activated or repressed in response to a change of an environmental condition, such as the change in concentration of a chemical, metal, radiation or nutrient or change in pH.

Furthermore, an inducible promoter useful in the methods and systems as disclosed herein can be a phage inducible promoter, nutrient inducible promoter, temperature inducible promoter, radiation inducible promoter, metal inducible promoter, hormone inducible promoter, steroid inducible promoter, and/or hybrids and combinations thereof. Promoters that are inducible by ionizing radiation can be used in certain embodiments, particularly in gene therapy of cancer, where gene expression is induced locally in the cancer cells by exposure to ionizing radiation such as UV or x-rays. Radiation inducible promoters include the non-limiting examples of fos promoter, c-jun promoter or at least one CArG domain of an Egr-1 promoter. Examples of inducible promoters include promoters from genes such as cytochrome P450 genes, heat shock protein genes, metallothionein genes, hormone-inducible genes, such as the estrogen gene promoter, and such.

In further embodiments, an inducible promoter useful in the methods and systems as disclosed herein can be Zn²⁺ metallothionein promoter, metallothionein-1 promoter, human metallothionein IIA promoter, lac promoter, lacO promoter, mouse mammary tumor virus early promoter, mouse mammary tumor virus LTR promoter, triose dehydrogenase promoter, herpes simplex virus thymidine kinase promoter, simian virus 40 early promoter or retroviral myeloproliferative sarcoma virus promoter. Other examples of inducible promoters include mammalian probasin promoter, lactalbumin promoter, GRP78 promoter, or the bacterial tetracycline-inducible promoter. Other examples include heat shock, steroid hormone, heavy metal, phorbol ester, adenovirus E1A element, interferon, and serum inducible promoters.

Inducible promoters useful in the methods and systems as disclosed herein for in vivo uses may include those responsive to biologically compatible agents, such as those that are usually encountered in defined animal tissues. An example is the human PAI-1 promoter, which is inducible by tumor necrosis factor. Further suitable examples cytochrome P450 gene promoters, inducible by various toxins and other agents; heat shock protein genes, inducible by various stresses; hormone-inducible genes, such as the estrogen gene promoter, and such. Inducible promoters may be inducible by Cu²⁺, Zn²⁺ tetracycline, tetracycline analog, ecdysone, glucocorticoid, tamoxifen, or an inducer of the lac operon (LacO). The promoter may be inducible by ecdysone, glucocorticoid, or tamoxifen. In specific embodiments, the inducible promoter is a phage inducible promoter, nutrient inducible promoter, temperature inducible promoter, radiation inducible promoter, metal inducible promoter, hormone inducible promoter, steroid inducible promoter, or combination thereof. Examples of radiation inducible promoters include fos promoter, jun promoter, or erg promoter.

An expression constructs may also contain a chemically inducible promoter (review article: Gatz et al. (1997) Annu Rev Plant Physiol Plant Mol Biol 48:89-108), by means of which the expression of the heterologous target gene can be controlled at a particular point in time. Such promoters such as, for example, a salicylic acid-inducible promoter (WO 95/19443), a benzenesulfonamide-inducible promoter (EP 0 388 186), a tetracycline-inducible promoter (Gatz et al. (1991) Mol Gen Genetics 227:229-237), an abscisic acid-inducible promoter EP 0 335 528) or an ethanol-cyclohexanone-inducible promoter (WO 93/21334) can likewise be used. Also suitable is the promoter of the glutathione-S transferase isoform II gene (GST-I I-27), which can be activated by exogenously applied safeners such as, for example, N,N-diallyl-2,2-dichloroacetamide (WO 93/01294) and which is operable in a large number of tissues of both monocotyledonous and dicotyledonous. Further exemplary inducible promoters that can be utilized in the instant invention include that from the ACE1 system which responds to copper (Mett et al. PNAS 90: 4567-4571 (1993)). An exemplary inducible promoter is the inducible promoter from a steroid hormone gene, the transcriptional activity of which is induced by a glucocorticosteroid hormone (Schena et al. (1991) Proc Nat'l Acad Sci USA 88:10421).

In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of secreted luciferase gene into RNA.

Viral expression vectors can be selected from a group comprising, for example, reteroviruses, lentiviruses, Epstein Barr virus-, bovine papilloma virus, adenovirus- and adeno-associated-based vectors, bacteriophages or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

Methods of delivering nucleic acid constructs as disclosed herein to the target cells (e.g., cells of the brain or other desired cancer target cells), can include, for example (i) injection of a composition containing the nucleic acid constructs, or (ii) directly contacting the cell, e.g., a cell of the brain, with a composition comprising the nucleic acid constructs, e.g., the nucleic acid construct can be injected directly into any blood vessel, such as vein, artery, venule or arteriole, via, e.g., hydrodynamic injection or catheterization. In some embodiments the nucleic acid constructs as disclosed herein can delivered to specific organs, for example the liver, bone marrow or systemic administration.

Administration can be by a single injection or by two or more injections. The nucleic acid constructs as disclosed herein can be delivered in a pharmaceutically acceptable carrier. One or more nucleic acid constructs as disclosed herein can be used simultaneously. The nucleic acid constructs as disclosed herein can be delivered singly, or in combination with other therapeutic agents, such as anti-cancer agents. The nucleic acid constructs as disclosed herein can also be administered in combination with other pharmaceutical agents which are used to treat or prevent cancers.

In one embodiment, specific cells are targeted with the nucleic acid constructs as disclosed herein, limiting potential side effects of the nucleic acid constructs caused by non-specific targeting of the nucleic acid constructs.

A viral-mediated delivery mechanism can also be employed the nucleic acid constructs as disclosed herein to cells in vitro and in vivo as described in Xia, H. et al. (2002) Nat Biotechnol 20(10):1006). Plasmid- or viral-mediated delivery mechanisms of the nucleic acid constructs as disclosed herein can also be employed to deliver the nucleic acid constructs to cells in vitro and in vivo as described in Rubinson, D. A., et al. ((2003) Nat. Genet. 33:401-406) and Stewart, S. A., et al. ((2003) RNA 9:493-501).

The nucleic acid constructs as disclosed herein can also be introduced into cells via the vascular or extravascular circulation, the blood or lymph system, and the cerebrospinal fluid.

The dose of the nucleic acid constructs as disclosed herein will be in an amount necessary to transducer the target cells and express the nucleic acid encoding the secreted luciferase.

The nucleic acid constructs as disclosed herein are useful for monitoring the progression of symptoms of a disease, such as cancer. In some embodiments, a measurable change in the level of the bioluminescence in a biological sample obtained from the subject from a second timepoint as compared to the level of the bioluminescence in a biological sample obtained from the subject from a first timepoint (i.e., a decrease in at least 10% or greater decrease in bioluminescent signal) indicates an improvement or a delay in the onset of a symptom in the subject and is indicative of an improved prognosis or attenuated progression of the disease, or where the effect of a anti-cancer agent or therapy is being monitored, is indicative of therapeutic efficacy of the agent.

### Formulations of Compositions

The nucleic acid constructs as disclosed herein can be used to monitor disease progression, for example progression of a cancer in a subject. The nucleic acid constructs as disclosed herein can be administered to cells in vivo, or in alternative embodiments to cells in culture, in vivo to cells in the body, or ex vivo to cells outside of an individual that can later be returned to the body of the same individual or another. Such cells can be disaggregated or provided as solid tissue.

Pharmaceutical compositions comprising the nucleic acid constructs as disclosed herein can be administered as a formulation adapted for passage through the blood-brain barrier or direct contact with the endothelium. In some embodiments, the compositions may be administered as a formulation adapted for systemic delivery. In some embodiments, the compositions can be administered as a formulation adapted for delivery to specific organs, for example but not limited to the liver, bone marrow, or systemic delivery.

Alternatively, pharmaceutical compositions can be added to the culture medium of cells ex vivo. In addition to the active compound, such compositions can contain pharmaceutically-acceptable carriers and other ingredients known to facilitate administration and/or enhance uptake (e.g., saline, dimethyl sulfoxide, lipid, polymer, affinity-based cell specific-targeting systems). The composition can be incorporated in a gel, sponge, or other permeable matrix (e.g., formed as pellets or a disk) and placed in proximity to the endothelium for sustained, local release. The composition can be administered in a single dose or in multiple doses which are administered at different times.

Pharmaceutical compositions can be administered by any known route. By way of example, the composition can be administered by a mucosal, pulmonary, topical, or other localized or systemic route (e.g., enteral and parenteral). The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of the agents as disclosed herein such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert.

The amount of the nucleic acid construct as disclosed herein which is administered to a subject is preferably an amount that does not induce toxic effects which outweigh the advantages which result from its administration. Further objectives are to transducer the tumor cells and allow secretion of the expressed secreted luciferase into the blood stream in order for it to be measurable in biological samples obtained from the subject.

Production of the nucleic acid constructs as disclosed herein for administration to subjects can be done according to present regulations will be regulated for good laboratory practices (GLP) and good manufacturing practices (GMP) by governmental agencies (e.g., U.S. Food and Drug Administration). This requires accurate and complete record keeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

### METHOD OF TO PERFORM ONE EMBODIMENT OF THE ASSAY

The following describes Materials and Methods useful not only for the studies that elucidated the use of a secretable form of luciferase to monitor disease progression, such as to monitor the size and progression of a tumor development, but also for the practice of the invention as described herein.

Reagents: A Gaussia luciferase-expressing plasmid which can be obtained from Nanolight (Pinetop, Az) or Targeting Systems (El Cajon, Calif.). A Lentivirus vector expressing Gluc which can be obtained from Targeting Systems (El Cajon, Calif.). In some embodiments, these constructs can also express a fluorescent protein such as GFP so one can monitor transduction efficiency by fluorescent microscopy. Coelenterazine, the Gaussia luciferase substrate, can be obtained from Nanolight (Pinetop, Az) or Targeting Systems (El Cajon, Calif.). Fetal bovine serum (FBS), penicillin and streptomycin mix, Dulbecco's modified Eagle's medium (DMEM) and OPTIMEM, 10× phosphate buffered saline (PBS) can be obtained from Cellgro by Mediatech Inc. (Hemdon, Va.). Tissue culture plates, can be obtained from Fisher Scientific (Pittsburgh, Pa.)

Equipment: DNA transduction efficiency is monitored using a confocal fluorescence microscope Zeiss LSM 510 (Jena, Germany). Luciferase can be measured by any equipment known by a person of ordinary skill in the art, for example, Gaussia luciferase activity can be measured using a luminometer (EG&G Berthold Microlumat). In vivo bioluminescene imaging can be carried out using a cooled couple-charged device (CCD) camera (Xenogen, Alamada, Calif.).

Procedure:

1) Gluc vector preparation. If one has a viral vector system which will efficiently transduced the specific cell type that they are interested in, one can subclone the Gluc cDNA into this vector using standard cloning protocols. In general, lentivirus vectors transduce many different cell types with high efficiency. The Gaussia luciferase cDNA, codon optimized for mammalian gene expression⁴, and a fluorescent protein such as GFP, separated by an internal ribosomal entry site (IRES) elements are cloned into a lentivirus vector under the control of the strong constitutive cytomegalovirus (CMV) promoter to produce LV-Gluc-GFP as described (Badr, et al., PLoS ONE 2, e571 (2007) and Sena-Esteves, et al. J Virol Methods 122, 131-139 (2004).

2) LV-Gluc-GFP transduction of cells of interest. Plate one million cells in a 2 cm² well and 24 hrs later, add lentivirus vector so you have a multiplicity of infection (MOI) of 10-50 in the presence of 8 μg/ml polybrene. After 5 h, wash the cells with PBS and add fresh culture medium and incubate the cells for 48 hrs. Alternatively, the cells of interest can be transfected by the plasmid expressing Gluc using transfection reagents such as lipofectamine (Invitrogen, Carlsbad, Calif.), though less efficient and transient. Next, determine the transduction/transfection efficiency by analysis of the CFP expression using fluorescence microscopy. In order to determine the Gluc expression, harvest 5-20 μl aliquots of the conditioned medium into a clean white or black 96-well plate, and measure the Gluc activity using a plate luminometer which was set to inject 50 μl 20 μM coelenterazine in DMEM and to acquire photon counts for 10 sec. If lentivirus vector was used to transduce the cells, the gene will integrate within the genome and therefore the cells become stably expressing the reporter, therefore, one may choose to grow and freeze the cells for later use.

3) Measurement of Gluc activity from cells-expressing it. Plate different amount of cells in a different well of a 96-well plate in triplicate. 24 hrs later, aliquot 10 μl of conditioned medium into a black or white plate and measure the Gluc activiy after injecting 50 μl 20 μM coelenterazine and acquire the signal for 10 sec using a luminometer. To monitor cell growth and proliferation, plate one to five thousand cells in a well of 96-well plate in triplicate and the next day, start taking 10 μl aliquots at different time points and measure the Gluc activity as above.

Note: Coelenterazine is known to auto-oxidizes. To stabilize this substrate, it should be diluted in DMEM and stored at R.T in the dark for 30 min before use. PBS with 5M NaCl could be used as an alternative diluent for coelenterazine to give higher light output and more stability of the substrate.

4) In vivo injection of Gluc-expressing cells. Anaesthetize the mice by i.p. injection of ketamine (100 mg/kg) and xylazine (5 mg/kg). Inject Gluc-expressing cells or Gluc-expression vectors at the location of interest or systemically (i.v.) and measure Gluc activity in the blood at several time-points before and after injection (see below).

5) Gluc blood monitoring. Blood samples are withdrawn by making a small incision in the tail of mice (no anesthesia required) or by retro-ocular withdrawal (anesthesia required). 1 blood is withdrawn using a p20 pipette and immediately added to 1μ Typically 5 μl 20 mM EDTA and stored at 4° C. until all samples are collected (up to 5 days). Gluc activity is measured using a plate luminometer which is set to inject 100 μl 100 μM coelenterazine (stabilized by incubated for 30 min at R.T.) in DMEM to 5 μl of blood samples and to acquire photon counts for 10 sec. As a background control, blood from mice injected with non-Gluc expressing cells is measured.

6) CCD camera imaging of Gluc signal. Mice are anesthetized as above and i.v. injected via tail-vein with 150 μl coelenterazine (4 mg/kg body weight, around 100 μg/mouse) and photon counts are acquired immediately over 1-5 min using a cooled CCD camera with no illumination as described (Tannous, et al., Mol Ther*11*8, 435-443 (2005). A light image of the animal is taken in the chamber using dim polychromatic illumination. Following data acquisition, post-processing and visualization is performed using CMIR-Image (a program developed by the Center for Molecular Imaging Research using image display and analysis suite developed in IDL (Research Systems Inc., Boulder, Colo.) or other programs available from the company from which the CCD camera was purchased. Regions of interest are defined using an automatic intensity contour procedure to identify bioluminescence signals with intensities significantly greater than the background. The mean, standard deviation, and sum of the photon counts in these regions are calculated as a measurement of Gluc activity. For visualization purposes, bioluminescence images are fused with the corresponding white light surface images in a transparent pseudocolor overlay, permitting correlation of areas of bioluminescent activity with anatomy.

In some embodiments, Coelenterazine may not be soluble in aqueous solution. If this is the case, then first, coelenterazine should be dissolved in acidified methanol (add a drop of concentration HCl to 10 ml of methanol) to a concentration of 5 g/l. Immediately before injection, mix 20 μl of coelenterazine with 130 μl PBS and i.v. inject it immediately. A small precipitate/cloudy solution might form during injection which normally does not interfere with imaging. However, if coelenterazine in PBS was incubated at room temperature (R.T.), a larger precipitate will form which might lead to blood-clot. Retro-ocular injections are normally easier to be done and one might choose to inject the substrate this way, rather than tail-vein injection. If this procedure is followed, one might observe some photons around the eye area which can be due to the way the injection was performed and this is normally fine.

In vivo studies. All animal studies should be performed with relevant institutional guidelines and regulations.

Read-Out of Results

In vitro detection of Gluc-expressing cells. Expect a linear curve of the Gluc signal with respect to cell number as well as with respect to time as an index of cell proliferation. For typical results, see Badr et al., (2007) PLoS ONE 2, e571.

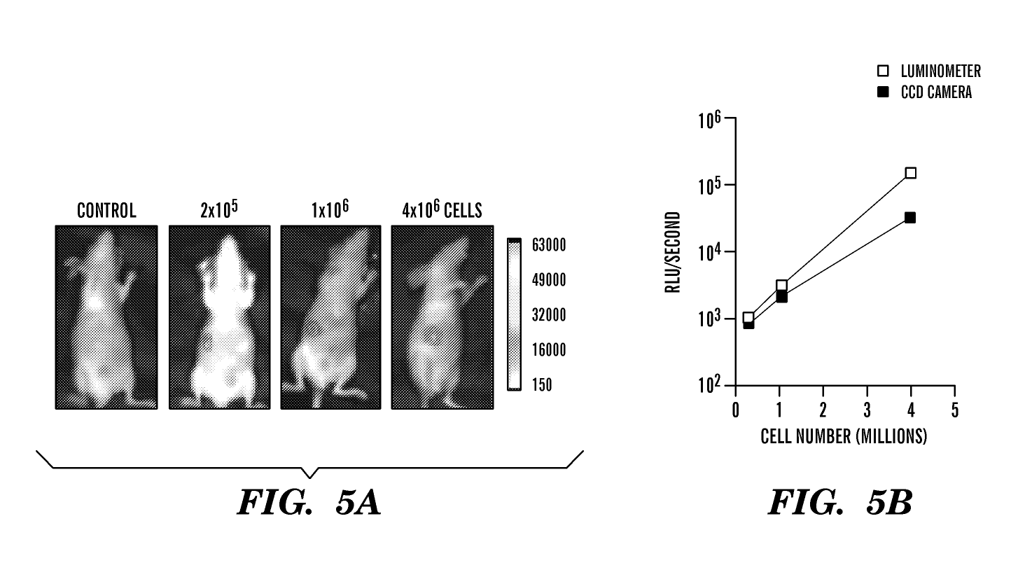

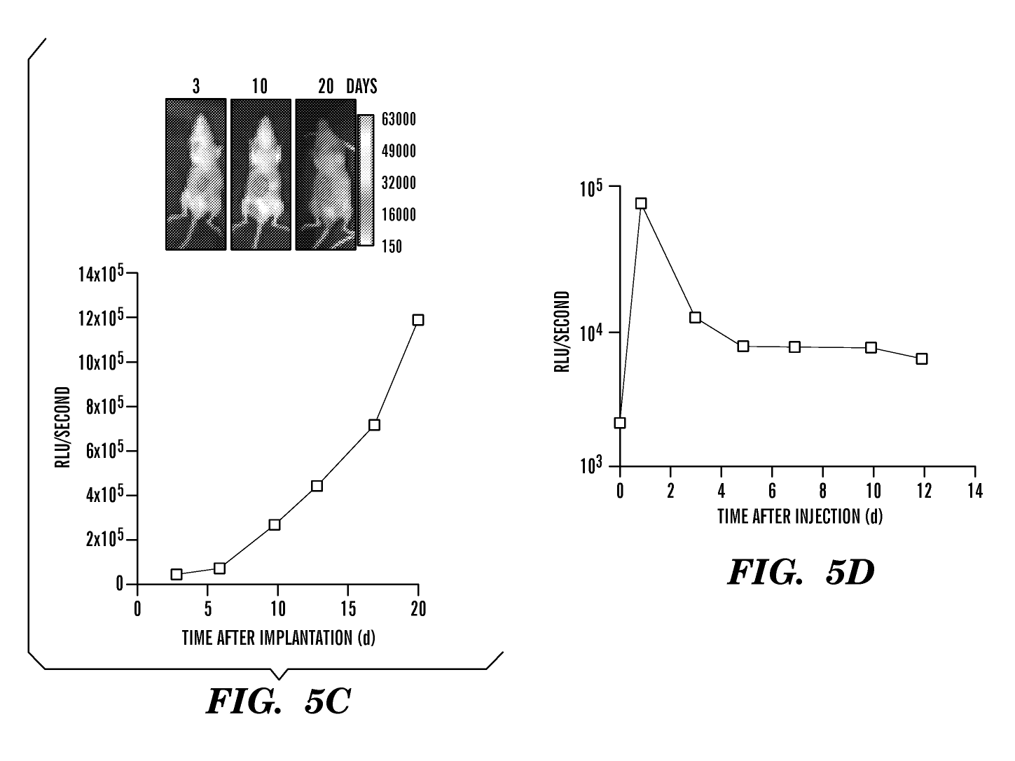

Monitoring of Gluc activity in vivo. Expect a linear curve between different amount of implanted cells and the Gluc signal obtained by either the blood assay or in vivo bioluminescence imaging using the CCD camera (FIGS. 5A & 5B). Also, expect an increase in the Gluc signal with respect to time (FIG. 5C).

Monitoring of circulating cells. Depending on the injected cell type, the results might vary. Initially, you would expect a drop in the Gluc signal since some of the injected cells are going to die. Later, the signal would either: remains the same indicating that the injected cells are still viable but not proliferating (FIG. 5D); the signal would increase indicating the cells are proliferating; the signal would decrease to background level indicating all cells are dead.

### Examples

The examples presented herein relate to the methods for monitoring severity of a cancer, for example by measuring size of a tumor by measuring the level of a secreted luciferase in a biological sample from the subject. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

Expression vectors. The Gaussia luciferase cDNA, codon optimized for mammalian gene expression, was cloned into a lentivirus vector under the control of the strong constitutive cytomegalovirus (CMV) promoter to produce LV-Gluc. In two other vectors, cDNAs for Gluc and the optimized cyan fluorescent protein (from Dr. David Piston, Vanderbilt Univ. Med. Ctr., TN) separated by IRES (LV-Gluc-CFP) or for SEAP (Clontech) and mCherry (from Dr. Roger Tsien, UCSD, CA) also separated by IRES (LV-SEAP-mCherry) were cloned under the CMV promoter. Lentivirus vector stocks were produced by triple transfection of 293T cells (provided by Dr. Michele Calos, Stanford Univ. Sch. Med.) with the lentivirus vector plasmid, the packaging genome plasmid, pCMVΔR8.91, and the plasmid, pVSV-G (Clontech) encoding the vesicular stomatitis virus envelope glycoprotein.

Cells. Gli36 cells (obtained from Dr. Anthony Campagnoni, UCLA, CA) were infected with 20 transducing units (TU)/cell of LV-Gluc to produce Gli36-Gluc. The C17.2 cell line is a v-myc immortalized mouse neuroprogenitor cell line (NPC; obtained from Dr. Evan Snyder, Burnham Institute, La Jolla Calif.). C17.2 cells were transduced with 20 tu/cell of LV-Gluc-CFP in order to produce NPC-Gluc-CFP cells.

Tumor models. All animal studies were approved by the Massachusetts General Hospital Review Board. For experimental brain tumors, nude mice were anesthetized with i.p. injection of ketamine (100 mg/kg) and xylazine (5 mg/kg) and then mounted on a stereotaxic frame using a specially made mould to orient the head. The skin was cleaned by scrubbing with 70% ethanol pads, followed by scrubbing with betadine pads. The skull was exposed by a small incision. A small burr hole (<0.5 mm) was made using a high-speed drill at the appropriate stereotaxic coordinates for injection into the frontal lobe of the brain. Two VI containing 1×10⁵ Gli36-Gluc cells was injected into the brain (2.5 mm lateral and 0.5 mm caudal to bregma; depth 3 mm from dura) with a 33 gauge Hamilton syringe. After closing the scalp the mice were placed on a warming pad and returned to cages after full recovery. For subcutaneous tumors, mice were anesthetized as above and different number of Gli36-Gluc or Gli36 expressing both Gluc and SEAP in 80 μl were pre-mixed with an equal volume of matrigel (BD Bioscience) and implanted in the flanks of nude mice.

Gluc blood assay. Blood samples were withdrawn by making a small incision in the tail of awake mice or by retro-ocular withdrawal from anesthetized mice. Typically, 5 μl blood was added to 1 μl 20 mM EDTA and Gluc activity was measured using a plate luminometer (EG & G Berthold Microlumat) which was set to inject 100 μl 100 μM coelenterazine in DMEM (Nanolight) and to acquire photon counts for 10 sec.

In vivo bioluminescence imaging. Mice were anesthetized as above and Gluc imaging was performed immediately after i.v. injection of 150 μl coelenterazine (4 mg/kg body weight) and recording photon counts over 5 min using a cooled CCD camera with no illumination9. A light image of the animal was taken in the chamber using dim polychromatic illumination. Following data acquisition, post-processing and visualization was performed using CMIR-Image, a program developed by the Center for Molecular Imaging Research using image display and analysis suite developed in IDL (Research Systems Inc., Boulder, Colo.). Regions of interest were defined using an automatic intensity contour procedure to identify bioluminescence signals with intensities significantly greater than the background. The mean, standard deviation, and sum of the photon counts in these regions were calculated as a measurement of Gluc activity. For visualization purposes, bioluminescence images were fused with the corresponding white light surface images in a transparent pseudocolor overlay, permitting correlation of areas of bioluminescent activity with anatomy.

Tissue Sectioning. Animals were sacrificed by transcardial perfusion with 4% paraformaldehyde in PBS under deep anesthesia. Tumors were removed, postfixed in paraformaldehyde and formalin/sucrose, frozen and sectioned into 7 μm sections. Sections were mounted on slides and evaluated for CFP expression using fluorescence microscopy.

### Example 1

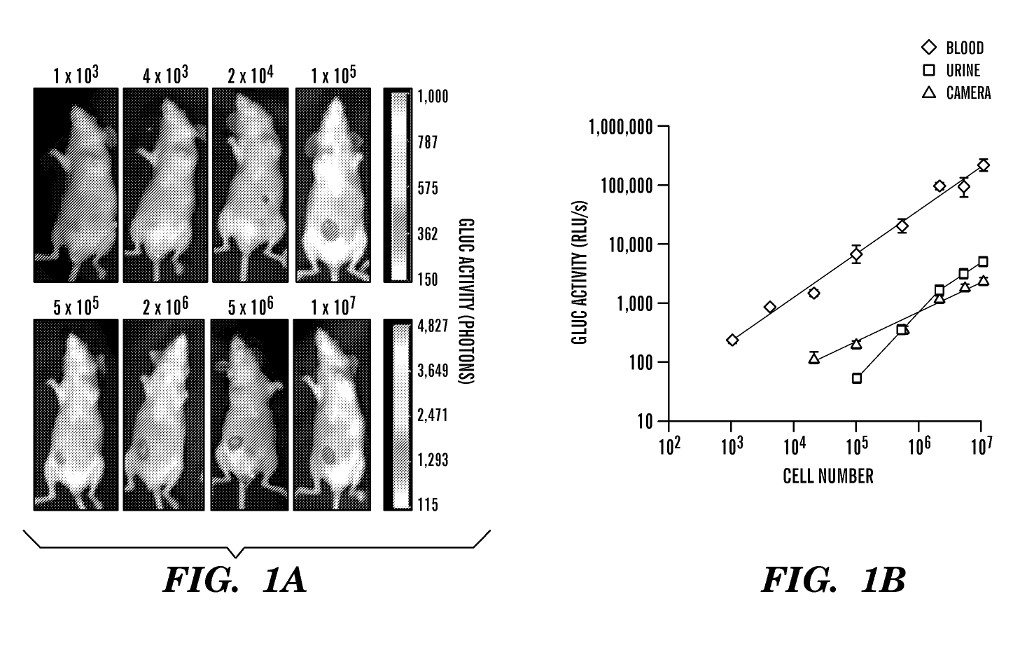

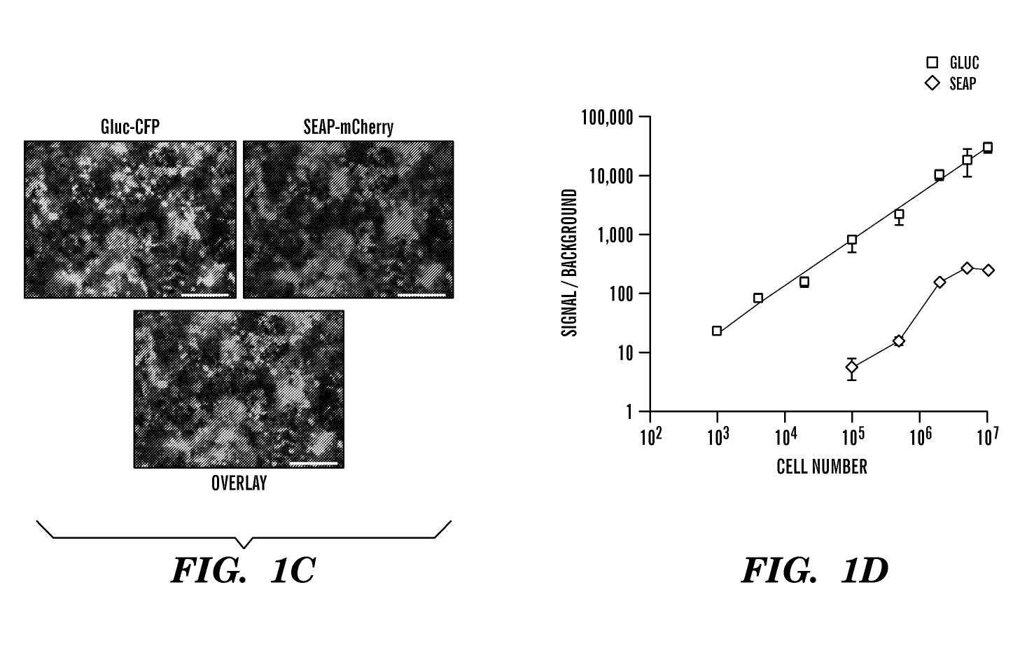

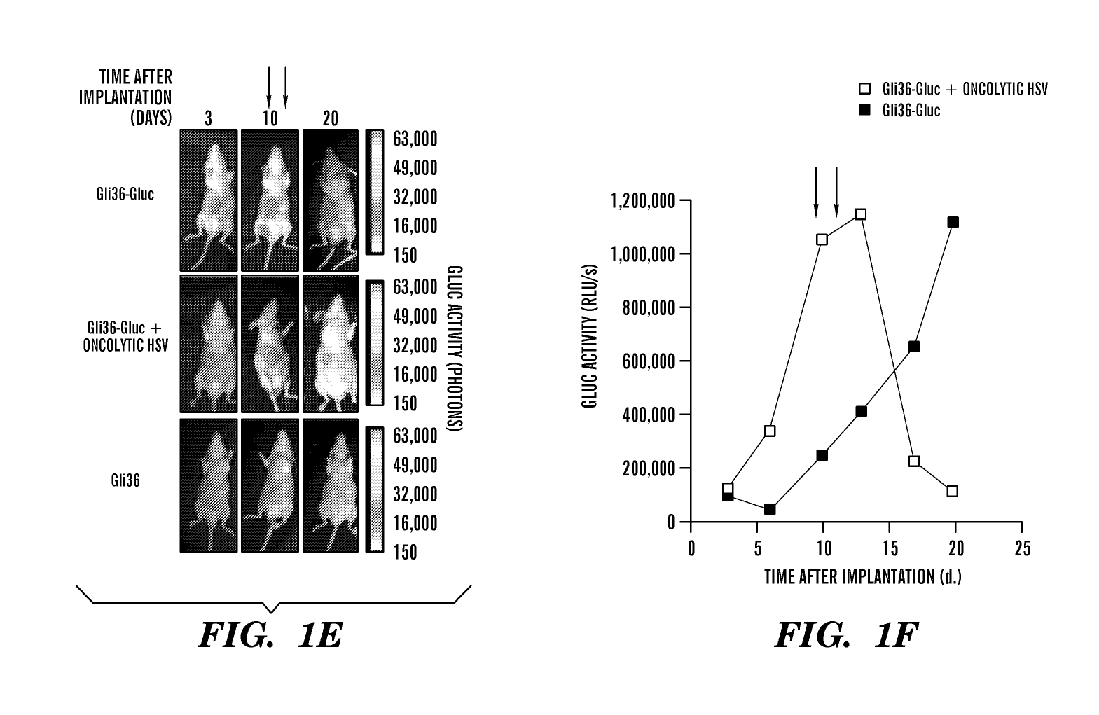

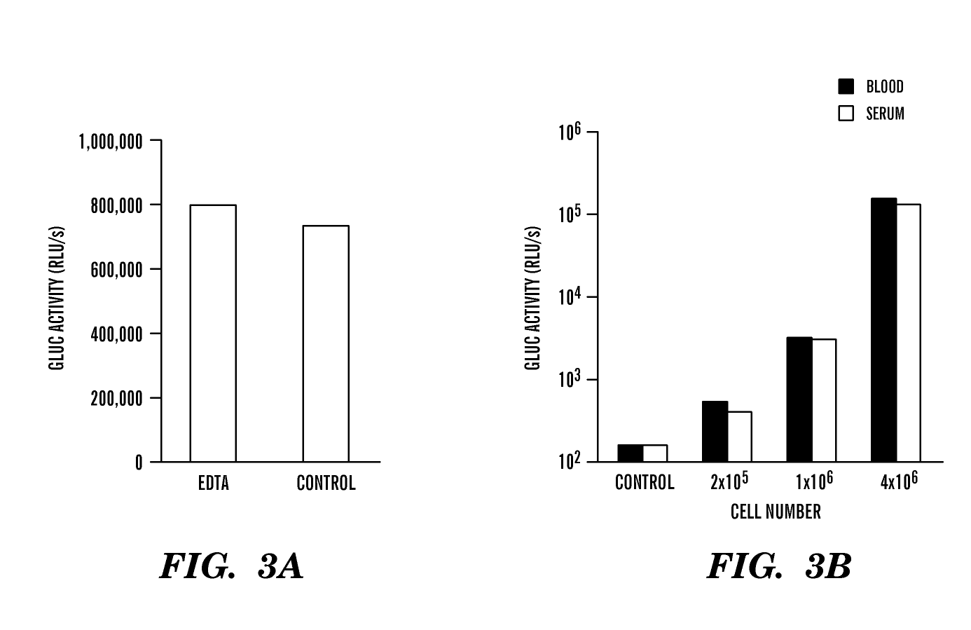

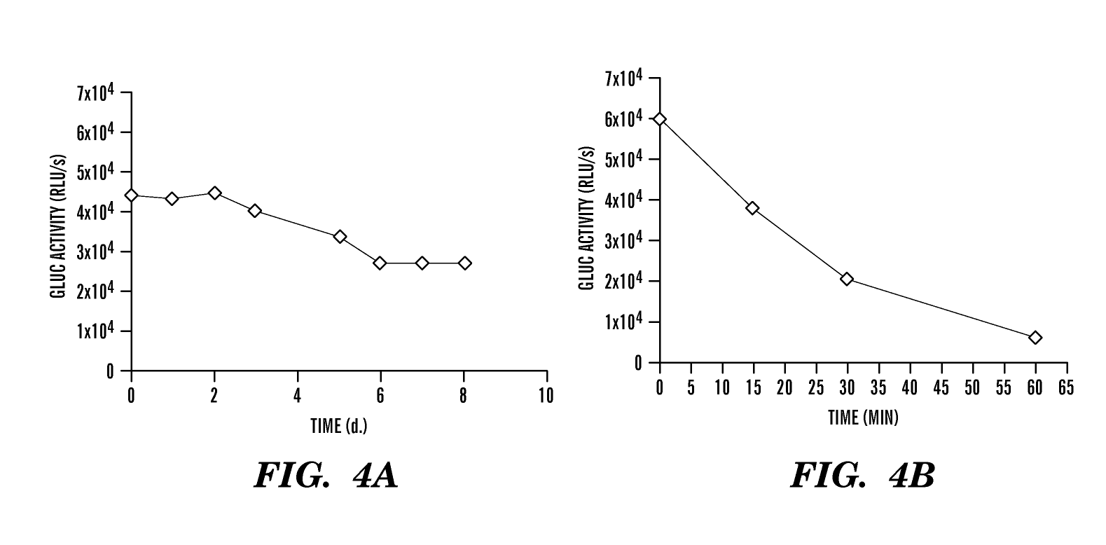

In order to assess the potential of Gluc as a reporter to monitor biological processes by measuring its level in the blood of small animals, the inventors transduced Gli36 human glioma cells with a lentivirus vector encoding Gluc (Gli36-Gluc) and implanted them in different numbers into the flanks of nude mice. The inventors visualized the tumors 3 days post-implantation by in vivo bioluminescence imaging after intravenous (i.v.) injection of the Gluc substrate, coelenterazine (4 mg/kg body weight) and acquiring photon counts using a CCD camera (FIG. 1a). At the same time, the inventors withdrew 5 μl blood samples from these mice and directly aliquoted them into tubes containing EDTA (1 μl 20 μM), after which the inventors measured the Gluc activity by adding coelenterazine (100 μM) and acquiring photon counts using a luminometer. The Gluc activity in the blood was linear with respect to cell number in a range covering over 5 orders of magnitude, and correlated well with values obtained using the CCD camera (FIG. 1B). Further, Gluc activity could also be detected in the urine, albeit to a lesser extent than in the blood, which indicates it is cleared by the kidneys (FIG. 1B). In addition, there was no detrimental effect of EDTA on Gluc activity measured in blood (FIG. 3A), and no significant differences were detected between the Gluc activity measured in serum or whole blood samples, showing that hemoglobin, which can interfere with luciferase measurement¹⁰, did not have a significant effect on Gluc activity under our assay conditions (FIG. 3B). Gluc samples could be stored at 4° C. for several days without significant decay of activity, with Gluc half-life being around 6 days (FIG. 4A). Further, the half-life of Gluc in the circulation in vivo is approximately 20 min (FIG. 4B), suggesting only a minor contribution of Gluc accumulation over time to the total Gluc signal measured in blood samples.

### Example 2

The inventors next compared the Gluc blood assay as disclosed herein to that of secreted alkaline phosphatase (SEAP), a well-established marker monitored in serum¹¹. The inventors co-infected (>90%) Gli36 cells with two different lentivirus vectors, one carrying the expression cassette for Gluc and cerulean fluorescent protein (CFP) separated by an internal ribosomal entry site (IRES) element, and the other a similar cassette encoding SEAP and mCherry, both driven by the CMV promoter (FIG. 1C). The inventors implanted different numbers of these cells subcutaneously in the flanks of nude mice and assayed the Gluc activity in 5 μL blood samples, as above, and SEAP activity in 5 μL serum as described¹¹. The Gluc blood assay was over 1000-fold more sensitive than the SEAP assay, being able to detect 1000 cells in vivo with signal/background (S/B) of 20, while the SEAP assay could only detect 500,000 cells with S/B of 10. Further the Gluc assay was linear with respect to cell number in a range covering over 5 orders of magnitude, while the SEAP assay fluctuated over 3 orders of magnitude with a plateau at around one million cells which could lead to greater underestimation of cell number. While the Gluc assay can be carried out with equal efficiency in blood or serum samples in few seconds, SEAP activity cannot be measured in blood since hemoglobin inhibits it. Also SEAP assay requires around 2 hrs handling prior to measurement. Gluc activity can be measured in urine, while at no time point was SEAP activity detected in the urine (data not shown). Gluc has a short half-life of 20 min in the blood allowing dynamic events to be monitored, whereas the half-life of SEAP is 3 h¹¹, leading to accumulation over time. Gluc can also be used to localize expressing cells in the animal by in vivo bioluminescence imaging, giving that sufficient cells are present at one location, while SEAP does not have this added advantage.

### Example 3

To determine whether Gluc activity in the blood could be used to monitor tumor growth and therapy in vivo, the inventors implanted nude mice subcutaneously with one million Gli36-Gluc cells. The inventors monitored tumor growth at different time points both with a CCD camera after i.v. injection of coelenterazine, or by assaying 5 μl blood samples for Gluc activity using a luminometer. At day 10 and 13 post-implantation, the inventors injected one set of mice intra-tumorally with an oncolytic HSV virus [hrR3, 108 plaque-forming unit (pfu)]¹² and a parallel control set with phosphate buffer saline (PBS). The Gluc signal in the blood from tumors treated with the oncolytic virus decreased dramatically, confirming its anti-tumor activity, while tumors treated with PBS increased logarithmically over time, with correlative changes in tumor volume in the CCD camera images (FIGS. 1E and 1F).

These results demonstrate that the Gluc levels in the blood can serve as a quantitative marker for the number of tumor cells expressing it in vivo, with complementary localization of the signal using a CCD camera.

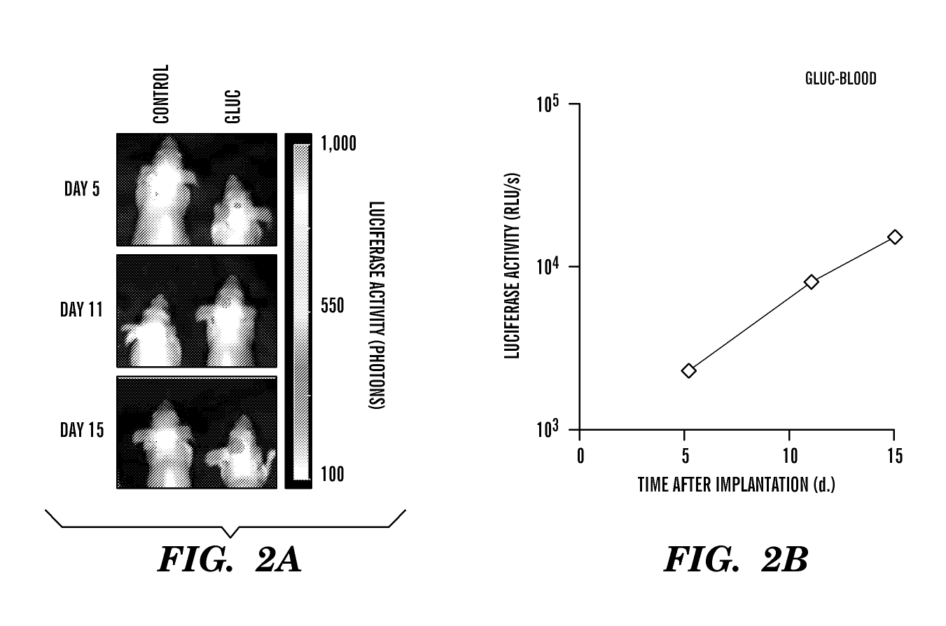

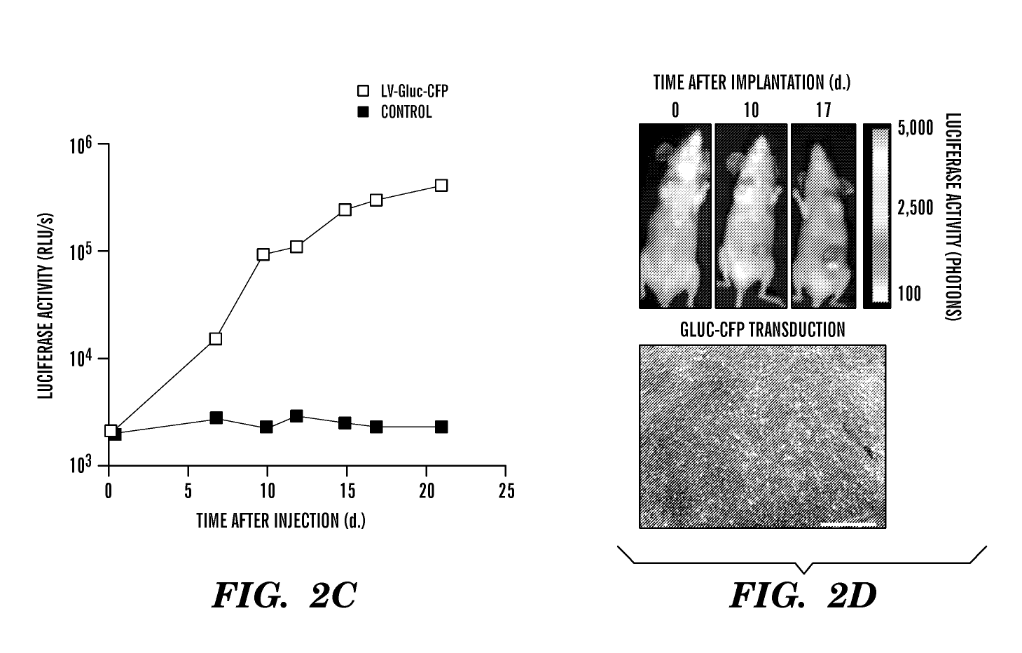

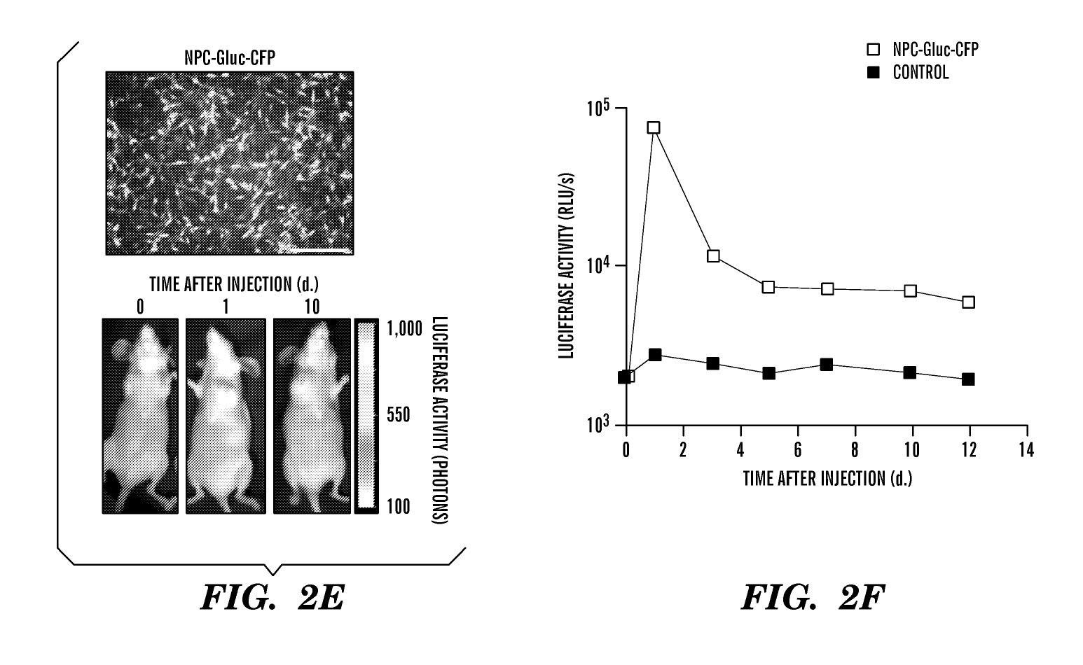

In order to evaluate the usefulness of the Gluc blood assay to monitor tumor growth in deep tissues, the inventors stereotactically injected one hundred thousand Gli36-Gluc cells into the brains of nude mice and monitored tumor volume over time using the CCD camera (FIG. 2A). At the same time, the inventors measured the levels of Gluc activity in the blood which showed increasing signal over time, indicating that Gluc is able to pass out through the brain tumor-barrier (BTB), allowing the easy monitoring of tumor growth in the brain from peripheral blood samples (FIG. 2B). The BTB is more permeable, however, than the blood brain-barrier, so Gluc exit from normal brain into the circulation might be more restricted.

To determine the usefulness of the Gluc blood assay to monitor gene transfer in vivo, the inventors implanted Gli36 tumors subcutaneously in nude mice and injected them with either a lentivirus vector carrying the expression cassette for Gluc and CFP or with PBS. A clear increase in the Gluc blood values was observed after viral transduction, indicating that stable gene transfer had occurred with inheritance to daughter cells (FIG. 2C). The Gluc signal was localized to the tumor by in vivo bioluminescence imaging and gene transfer was confirmed by analysis of tumor sections for CFP fluorescence (FIG. 2D). These results demonstrate that the Gluc blood assay can also be used to monitor gene transfer and proliferative fate of transduced tumor cells and thus serve as an index of gene therapy. This type of analysis could be extended for quantitative assessment of replication of virus vectors with applications in virology and vaccination.

### Example 4

In order to evaluate whether the Gluc blood assay could be used to monitor circulating cells in vivo, the inventors injected nude mice systemically (i.v.) with one million C17.2 neuronal precursor cells (NPCs) expressing Gluc and CFP (FIG. 2e) and monitored Gluc activity in 20 μl blood over time. Mice injected with NPC-Gluc cells showed an initial high Gluc value which decreased after 3 days, indicating that a significant number of the NPCs survived the injection procedure. By four days post-injection the level of Gluc stabilized, indicating that the surviving cells were maintained but did not proliferate (FIG. 2F). The inventors were not able to localize a Gluc signal by CCD camera imaging anywhere in the body, demonstrating that the NPC cells did not concentrate in any one tissue (FIG. 2E). These results demonstrate that the Gluc blood assay can be used to monitor circulating cell viability in vivo and, as such, could be extended to monitor stem cell transplantation.

Here, the inventors have discovered a novel use for the Gaussia luciferase (Gluc) reporter as a tool for quantitative assessment of different biological processes in small animals by measuring its level in the blood. The Gluc blood assay was demonstrate to be useful in monitoring tumor growth and therapy, gene transduction as well as circulating cell survival and can be readily extended to many other applications involving luciferase-mediated bioluminescence. The Gluc blood assay provides a sensitive and quantitative assessment of numbers of transduced cells in vivo, complementing in vivo bioluminescence imaging which has the unique ability to localize the signal, and thereby greatly facilitating non-invasive monitoring of numerous biological processes.

### REFERENCES

The references cited herein and throughout the application are incorporated herein by reference.
1 Contag, C. H. & Ross, B. D. It's not just about anatomy: in vivo bioluminescence imaging as an eyepiece into biology. J Magn Reson Imaging 16, 378-387 (2002).
2 Bhaumik, S. & Gambhir, S. S. Optical imaging of Renilla luciferase reporter gene expression in living mice. Proc Natl Acad Sci USA 99, 377-382 (2002).
3 Gross, S. & Piwnica-Worms, D. Spying on cancer: molecular imaging in vivo with genetically encoded reporters. Cancer cell 7, 5-15 (2005).
4 Negrin, R. S. & Contag, C. H. In vivo imaging using bioluminescence: a tool for probing graft-versus-host disease. Nat Rev Immunol 6, 484-490 (2006).
Adams, J. Y. et al. Visualization of advanced human prostate cancer lesions in living mice by a targeted gene transfer vector and optical imaging. Nat Med 8, 891-897 (2002).
6 Luker, G. D. et al. Noninvasive bioluminescence imaging of herpes simplex virus type 1 infection and therapy in living mice. J Virol 76, 12149-12161 (2002).
7 Luo, J., Lin, A. H., Masliah, E. & Wyss-Coray, T. Bioluminescence imaging of Smad signaling in living mice shows correlation with excitotoxic neurodegeneration. Proc Natl Acad Sci USA 103, 18326-18331 (2006).
8 Badr, C. E., Hewett, J. W., Breakefield, X. O. & Tannous, B. A. A highly sensitive assay for monitoring the secretory pathway and ER stress. PLoS ONE 2, e571 (2007).
9 Tannous, B. A., Kim, D. E., Fernandez, J. L., Weissleder, R. & Breakefield, X. O. Codon-optimized Gaussia luciferase cDNA for mammalian gene expression in culture and in vivo. Mol Ther 11, 435-443 (2005).
10 Colin, M. et al. Haemoglobin interferes with the ex vivo luciferase luminescence assay: consequence for detection of luciferase reporter gene expression in vivo. Gene therapy 7, 1333-1336 (2000).
11 Hiramatsu, N., Kasai, A., Hayakawa, K., Yao, J. & Kitamura, M. Real-time detection and continuous monitoring of ER stress in vitro and in vivo by ES-TRAP: evidence for systemic, transient ER stress during endotoxemia. Nucleic Acids Res 34, e93 (2006).
12 Kramm, C. M. et al. Herpes vector-mediated delivery of marker genes to disseminated central nervous system tumors. Hum Gene Ther 7, 291-300 (1996).
13 Weissleder, R. A clearer vision for in vivo imaging. Nat Biotechnol 19, 316-317 (2001).

1. An assay for identifying an agent which modulates cell growth comprising:
a. measuring the bioluminescent signal from a cell expressing a Gaussia luciferase protein at a first timepoint;
b. contacting the cells with at least one agent;
c. measuring the bioluminescent signal from the cell expressing Gaussia luciferase protein at a second timepoint;
wherein a change in the bioluminescent signal measured from the Gaussia luciferase protein at the second time point as compared to the first timepoint identifies an agent which modulates cell growth.

2. The assay of claim 1, wherein modulation is an increase in cell growth.

3. The assay of claim 1, wherein modulation is a decrease in cell growth.

4. The assay of claim 1, wherein the cell is in a population of cells.

5. The assay of claim 1, wherein the cell is a proliferating cell.

6. The assay of claim 5, wherein the proliferating cell is a tumor cell.

7. The assay of claim 1, wherein the cell is present within a subject or in vivo.

8. The assay of claim 7, wherein the subject is an animal model of cancer.

9. The assay of claim 7, wherein the bioluminescent signal from the Gaussia luciferase protein is measured in a biological sample obtained from the subject.

10. The assay of claim 9, wherein the biological sample is selected from the group consisting of: urine, blood, plasma, lymph fluid, cerebrospinal fluid.

11. The assay of claim 7, wherein the bioluminescent signal from the Gaussia luciferase protein is measured within the subject.

12. The assay of claim 1, wherein the step of measuring the bioluminescent signal from the Gaussia luciferase protein at a second timepoint is performed at several different time points.

13. The assay of claim 1, wherein the contacting a cell with at least one agent is for a sufficient amount of time for modulation of the cell growth to occur.

14. The assay of claim 1, wherein the Gaussia luciferase protein is encoded by the nucleic acid sequence of SEQ ID NO: 1 or a variant or fragment thereof.

15. The assay of claim 1, wherein the Gaussia luciferase protein is encoded by a Gaussia luciferase nucleic acid sequence which is codon optimized for mammalian gene expression.

16. The assay of claim 14, wherein the Gaussia luciferase nucleic acid sequence is humanized Gaussia luciferase (hGLuc) and has the nucleic acid sequence as set forth in SEQ ID NO:3.

17. The assay of claim 1, wherein the cell expressing the Gaussia luciferase protein comprises a nucleic acid construct comprising a nucleic acid encoding the Gaussia luciferase operatively linked to a promoter.

18. The assay of claim 17, wherein the promoter is a tissue specific promoter, or a cancer specific promoter.

19. The assay of claim 17, wherein the promoter is a constitutively active promoter.

20. The assay of claim 17, wherein the promoter is an inducible promoter.

21. The assay of claim 17, wherein the nucleic acid construct is a vector.

22. The assay of claim 17, wherein the nucleic acid construct is an expression vector.

23. The assay of claim 17, wherein the vector is a viral vector.

24. The assay of claim 13, wherein the viral vector is selected from a group consisting of: a lentivirus vector, a retroviral vector, a herpes simplex viral vector, an adenovirus vector, an adeno-associated virus (AAV) vector, an EPV vector, an EBV vector and a bacteriophage vector.

25. A cell comprising a nucleic acid construct comprising a nucleic acid encoding a Gaussia luciferase protein operatively linked to a promoter.

26. The cell of claim 25, wherein the Gaussia luciferase protein is encoded by the nucleic acid sequence of SEQ ID NO: 1 or a variant or fragment thereof.

27. The cell of claim 25, wherein the Gaussia luciferase protein is encoded by a Gaussia luciferase nucleic acid sequence which is codon optimized for mammalian gene expression.

28. The cell of claim 27, wherein the Gaussia luciferase nucleic acid sequence is humanized Gaussia luciferase (hGLuc) and has the nucleic acid sequence as set forth in SEQ ID NO:3.

29. An animal comprising a cell of any of claims 25 to 28.

30. The animal of claim 29, wherein the animal is a transgenic animal.

31. The animal of claim 29, wherein animal has been transplanted with the cell comprising a nucleic acid construct comprising a nucleic acid encoding a Gaussia luciferase protein operatively linked to a promoter.

32. The animal of claims 29, wherein the animal is a small experimental animal.

33. The animal of claim 32, wherein the small experimental animal is a rodent.


SEQUENCE LISTING (4 sequences)

SEQ ID NO: 1 - DNA, 758 bp, Gaussia princeps
ggtactcaaagtatcttctggcagggaaaatgggagtgaaagttctttttgcccttatttgtattgctgtggccgaggccaaaccaactgaaaacaatgaagatttcaacattgtagctgtagctagcaactttgctacaacggatctcgatgctgaccgtggtaaattgcccggaaaaaaattaccacttgaggtactcaaagaaatggaagccaatgctaggaaagctggctgcactaggggatgtctgatatgcctgtcacacatcaagtgtacacccaaaatgaagaagtttatcccaggaagatgccacacctatgaaggagacaaagaaagtgcacagggaggaataggagaggctattgttgacattcctgaaattcctgggtttaaggatttggaacccatggaacaattcattgcacaagttgacctatgtgtagactgcacaactggatgcctcaaaggtcttgccaatgtgcaatgttctgatttactcaagaaatggctgccacaaagatgtgcaacttttgctagcaaaattcaaggccaagtggacaaaataaagggtgccggtggtgattaatcctaatagaatactgcataactggatgatgatatactagcttattgctcataaaatggccattttttgtaacaaatcgagtctatgtaattcaaaatacctaattaattgttaatacatatgtaattcctataaatataatttatgcaatccaaaaaaaaaaaaaaaaaa

SEQ ID NO: 2 - protein, 185 aa, Gaussia princeps
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 3 - DNA, 558 bp, Gaussia princeps
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactag

SEQ ID NO: 4 - protein, 185 aa, Gaussia princeps; identical to SEQ ID NO: 2